(19) United States
(12) Patent Application Publication
(10) Pub. No.: US 2008/0096868 A1
(43) Pub. Date: Apr. 24, 2008

(54) 1,4 Substituted Pyrazolopyrimidines as Kinase Inhibitors

(76) Inventors: Niko Schmiedeberg, Basel (CH); Pascal Furet, Thann (FR); Patricia Imbach, Kaiseraugst (CH); Philipp Holzer, Suhr (CH)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 11/718,730

(22) PCT Filed: Nov. 10, 2005
(86) PCT No.: PCT/EP05/12045

§ 371 Date: May 7, 2007

(30) Foreign Application Priority Data
Nov. 12, 2004 (GB) 0425035.3

(51) Int. Cl. A61K 31/519; A61K 31/5377; A61K 31/551; A61P 35/00; C07D 487/04

(52) U.S. Cl. 514/218; 514/234.2; 514/252.16; 514/262.1; 540/575; 544/118; 544/262

(57) ABSTRACT

The invention relates to 1,4-substituted pyrazolopyrimidine compounds of the formula I, 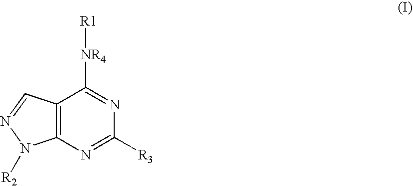 pharmaceuticals comprising a 1,4-substituted pyrazolopyrimidine compound, the use of a 1,4-substituted pyrazolopyrimidine compound in the treatment or the use thereof in the manufacture of a pharmaceutical formulation for the treatment of a disease that depends on inadequate activity of a protein kinase, methods of treatment comprising administering a 1,4-substituted pyrazolopyrimidine compound, methods for the manufacture of a novel compound of that class, and novel intermediates and partial steps for their synthesis.

The invention relates to 1,4-substituted pyrazolopyrimidines for use in the diagnostic and therapeutic treatment of a warm-blooded animal, especially for the treatment of a disease (=disorder) that depends on inadequate activity of a protein kinase; the use of a compound of that class for the preparation of a pharmaceutical formulation for the treatment of a disease that depends on inadequate activity of a protein kinase; the use of a compound of that class in the treatment of a disease that depends on inadequate activity of a protein kinase; novel 1,4-substituted pyrazolopyrimidine compounds, pharmaceuticals comprising a 1,4-substituted pyrazolopyrimidine compound, the use of a 1,4-substituted pyrazolopyrimidine compound in the treatment or the use thereof in the manufacture of a pharmaceutical formulation for the treatment of a disease that depends on inadequate activity of a protein kinase, methods of treatment comprising administering a 1,4-substituted pyrazolopyrimidine compound, methods for the manufacture of a novel compound of that class, and novel intermediates and partial steps for their synthesis.

Certain 4-substituted hydrazono pyrazolopyrimidines have been described for use as GSK3 kinase inhibitors in the treatment of e.g. diabetes and TIE-2 kinase related diseases, see WO 04/009602, WO 04/009596 or WO 04/009597. On the other hand, certain acyl- or acylamino-substituted arylamino-pyrazolopyrimidines have been described as p38-inhibitors, see WO 03/099280.

Over the past years, basic roles for Eph receptor tyrosine kinases and their ligands, the ephrins, could be established. Fourteen different Eph receptors were catalogued and grouped into EphA or EphB subclasses, based on their affinity for ligands. Eight ephrins have been identified which are membrane proteins, either of the glycerophosphatidylinositol (GPI)-linked (ephrinA) or transmembrane (ephrinB) type. Signaling between Eph receptors and their ligands is restricted to sites of direct cell-cell contact. The result of contact is the induction of reciprocal bidirectional events between cells. The expression of ephrins and their receptors at certain locations is presumed to have impact on tissue patterning and the organizing of spatially very restricted cell loci. Among the specific effects are the modification of cell migration, adhesion and somite formation.

EphB4 (also named HTK) and its ligand, ephrinB2 (HTKL) have critical roles in establishing and determining vascular networks. On the venous epithelium, EphB4 is expressed specifically, while, during early stages of vascular development, ephrinB2 is specifically and reciprocally expressed on arterial endothelial cells. Dysfunctional genes lead to embryonic lethality in mice, and the embryos show identical defects in forming capillary connections in case of either defect ephrinB2 and EphB4. Both are expressed at the first site of hematopoiesis and vascular development during embryogenesis. An essential role for proper hematopoietic, endothelial, hemangioblast and primitive mesoderm development was established. EphB4 deficiency results in an alteration in the mesodermal differentiation outcome of embryonic stem cells. Ectopic expression of EphB4 in mammary tissue results in disordered architecture, abnormal tissue function and a predisposition to malignancy (see e.g. N. Munarini et al., J. Cell. Sci. 115, 25-37 (2002)). From these and other data, it has been concluded that inadequate EphB4 expression may be involved in the formation of malignancies and thus that inhibition of EphB4 can be expected to be a tool to combat malignancies, e.g. cancer and the like.

The conversion of the abl proto-oncogene into an oncogene has been observed in patients with chronic myelogenous leukemia (CML). A chromosome translocation joins the bcr gene on chromosome 22 to the abl gene from chromosome 9, thereby generating a Philadelphia chromosome. The resulting fusion protein has the amino terminus of the Bcr protein joined to the carboxy terminus of the Abl tyrosine protein kinase. In consequence, the Abl kinase domain becomes inappropriately active, driving excessive proliferation of a clone of hematopoietic cells in the bone marrow. Inhibition of this tyrosine kinase by the active principle of Gleevec™ or Glivec® (trademarks of Novartis), an inhibitor of this fusion protein, has been shown to be a highly active treatment against CML. Thus the general concept that inadequate expression of Abl tyrosine kinase can remedy malignancies, especially leukemias, could be exemplified.

The constitutively expressed viral form c-Src (from Rous Sarcoma Virus, a retrovirus) of the tyrosine kinase c-Src found in cells is an example how inadequate expression of the Src protein tyrosine kinase can lead to malignancies based on transformed cells. Inhibition of Src protein tyrosine kinase can lead to inhibition of deregulated growth of the transformed tumor cells, e.g. in connective-tissue tumors. Therefore, also here inhibition of c-Src or modified or mutated forms thereof is expected to show a beneficial effect in the treatment of proliferative diseases.

This leads to the problem of the present invention: In view of the large number of protein kinase inhibitors and the multitude of proliferative and other protein kinase-related diseases, there is an ever-existing need to provide new classes of compounds that are useful as protein kinase inhibitors and thus in the treatment of these protein tyrosine kinase, such as serine/threonine and/or preferably PTK (protein tyrosine kinase) related diseases. What is required are new classes of pharmaceutically advantageous protein kinase, especially PTK inhibiting compounds, especially with advantageous properties, such as high affinity and/or selectivity for limited groups of or singular protein kinases.

The present invention is based on the unexpected finding that the 1,4-substituted pyrazolopyrimidines of the formula I given below show activity at least, preferably selectively, on one or more protein kinases, such as of the kinases mentioned below, especially those mentioned as preferred. These compounds can thus be used as basis for potent medications. In addition, they show further advantageous pharmaceutically useful properties, especially a good selectivity for certain protein kinases as defined below.

It has been found that the members of a novel class of 1,4-substituted pyrazolopyrimidine compounds of the formula I described below are inhibitors of specific types or classes or groups of protein kinases, especially PTK, such as preferably one or more of c-Abl, c-Src and/or especially Ephrin receptor kinase, especially EphB4 kinase; and/or one or more altered or mutated forms of any one or more of these (e.g. those that result in conversion of the respective proto-oncogene into an oncogene, such as constitutively activated Bcr-Abl or v-Src). In view of these activities, the compounds can be used for the treatment of diseases related to inadequate, especially aberrant or excessive, activity of such types of kinases, especially those mentioned above and most especially those mentioned as being preferred.

The invention in particular relates to 1,4-substituted pyrazolopyrimidine compounds of the formula I, 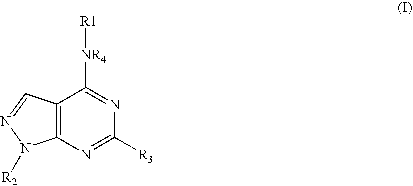
wherein
R₁ is a moiety of the formula Ib 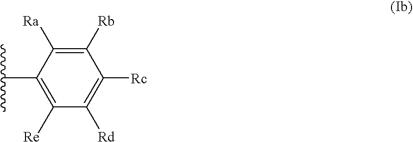
wherein Ra is methyl, ethyl, methoxy, halo or trifluoromethyl;
Re is hydrogen, methyl, ethyl, methoxy, halo or trifluoromethyl, and
Rb, Rc and Rd are independently selected from hydrogen and phenyl substitutents;
R₂ is unsubstituted or substituted aryl;
R₃ is hydrogen, unsubstituted or substituted alkyl, unsubstituted or substituted aryl or unsubstituted or substituted heterocyclyl; and
R₄ is hydrogen or unsubstituted or substituted alkyl;
or a (preferably pharmaceutically acceptable) salt thereof where one or more salt-forming groups are present, for use in the diagnostic or preferably therapeutic treatment of a warm-blooded animal, especially for use in the treatment of a disease or disorder that is dependent on inadequate activity of a protein kinase, especially a protein tyrosine kinase, especially of one or more of c-Abl, c-Src and/or especially an Ephrin receptor kinase, especially EphB4 kinase; and/or one or more altered or mutated forms of any one or more of these (e.g. those that result in conversion of the respective proto-oncogene into an oncogene, such as constitutively activated Bcr-Abl or v-Src).

The invention, in a further and preferred embodiment, relates to the use of compounds of a compound of the formula I, or a pharmaceutically acceptable salt thereof, in the pre-paration of a pharmaceutical formulation for the treatment of a disease or disorder that depends on inadequate activity of a protein kinase, especially a protein tyrosine kinase, especially one or more of c-Abl, c-Src and/or especially Ephrin receptor kinase, especially EphB4 kinase; and/or one or more altered or mutated forms of any one or more of these (e.g. those that result in conversion of the respective proto-oncogene into an oncogene, such as constitutively activated Bcr-Abl or v-Src), or the use of said compounds in the treatment of a disease that depends on inadequate activity of a protein kinase, especially a protein tyrosine kinase, especially as defined above.

Yet another embodiment of the invention relates to a novel 1,4-substituted pyrazolopyrimidine compound of the formula I given above, wherein
R₁ is a moiety of the formula Ib 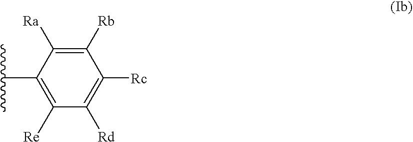
wherein Ra is methyl, ethyl, methoxy, halo or trifluoromethyl;
Re is hydrogen, methyl, ethyl, methoxy, halo or trifluoromethyl, and
Rb, Rc and Rd are independently selected from hydrogen, unsubstituted or substituted alkyl, unsubstituted or substituted alkenyl, unsubstituted or substituted alkynyl, unsubstituted or substituted aryl, unsubstituted or substituted heterocyclyl, hydroxy, esterified or etherified hydroxy, unsubstituted, mono- or disubstituted amino wherein the substitutents are independently selected from unsubstituted or substituted alkyl and unsubstituted or substituted aryl; halo, nitro, cyano, mercapto, substituted mercapto, sulfo and substituted sulfonyl wherein the substituents are selected from unsubstituted or substituted alkyl and unsubstituted or substituted aryl;

R₂ is unsubstituted or substituted aryl;

R₃ is hydrogen, unsubstituted or substituted alkyl, unsubstituted or substituted aryl or unsubstituted or substituted heterocyclyl; and

R₄ is hydrogen or unsubstituted or substituted alkyl,

with the proviso that if R₂ is 4-methoxyphenyl, R₃ is hydrogen and R₄ is hydrogen, then R₁ is other than 5-fluoro-2-methylphenyl and 2-methylphenyl; and with the proviso that R₁ is other than unsubstituted or substituted 3-nitrophenyl;

or a (preferably pharmaceutically acceptable) salt thereof.

Another embodiment of the invention relates to a compound of the formula I, wherein

R₁ is 5-fluoro-2-methylphenyl and 2-methylphenyl

R₂ is 4-lower alkoxyphenyl,

R₃ is hydrogen,

R₄ is hydrogen;

or a pharmaceutically acceptable salt thereof, for use in the diagnostic or preferably therapeutic treatment of a warm-blooded animal, especially for use in the diagnostic and therapeutic treatment of a disease that depends on inadequate activity of a protein kinase, preferably a protein tyrosine kinase, and/or the use of such a compound for the manufacture of a pharmaceutical formulation for the treatment of a disease or disorder that depends on inadequate protein kinase, especially tyrosine kinase, activity, especially of one or more of the tyrosine kinases mentioned herein as preferred.

Still another embodiment of the invention relates to a compound of the formula I,

wherein R₁ is unsubstituted or substituted 3-nitrophenyl;

R₂ is substituted aryl;

R₃ is hydrogen or unsubstituted or substituted alkyl; and

R₄ is hydrogen or unsubstituted or substituted alkyl,

or a pharmaceutically acceptable salt thereof;

for use in the diagnostic or preferably therapeutic treatment of a warm-blooded animal, especially for use in the diagnostic and therapeutic treatment of a disease that depends on inadequate activity of a protein kinase, especially a protein tyrosine kinase.

Another embodiment of the invention relates to a pharmaceutical formulation comprising a 1,4-substituted pyrazolopyrimidine compound of the formula I, especially a novel compound of the formula I, or a pharmaceutically acceptable salt thereof, especially useful in the treatment of a disease or disorder that depends on inadequate activity of a protein kinase, especially a protein tyrosine kinase.

Where a protein kinase is mentioned, this relates to any type of protein kinase, especially serine/threonine and/or preferably protein tyrosine kinases, such as protein kinase C, c-Abl, Bcr-Abl, c-Kit, c-Raf, Fit-1, Flt-3, PDGFR-kinase, c-Src, FGF-R1, FGF-R2, FGF-R3, FGF-R4, casein kinases (CK-1, CK-2, G-CK), Pak, ALK, ZAP70, Jak1, Jak2, Axl, Cdk1, cdk4, cdk5, Met, FAK, Pyk2, Syk, Insulin receptor kinase, Tie-2 or constitutively activating mutations of kinases (activating kinases) such as of Bcr-Abl, c-Kit, c-Raf, Flt-3, FGF-R3, PDGF-receptors, VEGF-receptors, S-1P, IGF-1 receptor, and/or Met, and/or one or more altered or mutated forms of any one or more of these. Preferred are protein tyrosine kinases.

Where a protein (especially tyrosine) kinase is mentioned hereinbefore and hereinafter, this relates preferably to one or more of c-Abl, c-Src and/or especially Ephrin receptor kinase, especially EphB4 kinase; and/or one or more altered or mutated forms of any one or more of these (e.g. those that result in conversion of the respective proto-oncogene into an oncogene, such as constitutively activated Bcr-Abl or v-Src), if not mentioned otherwise.

The invention also, in still other embodiments, relates to the use of a (preferably novel) compound of the formula I in the treatment or the use thereof in the manufacture of a pharmaceutical formulation for the treatment of a disease that depends on inadequate activity of a protein kinase, especially a tyrosine protein tyrosine kinase; to a method of treatment as defined above for all compounds of the formula I comprising administering a novel 1,4-substituted pyrazolopyrimidine compound of the formula I, or a pharmaceutically acceptable salt thereof; and/or to a method for the manufacture of the novel compounds of the formula I, and novel intermediates and partial steps for the synthesis of a compound of the formula I.

The general terms or symbols used hereinbefore and hereinafter preferably have, within the context of this disclosure, the following meanings, unless otherwise indicated:

In each case where a waved line vertical to a bond is used, this marks the end of that bond via which a given moiety is bound to the rest of the corresponding molecule.

The term “lower” or “C₁-C₇-” defines a moiety with up to and including maximally 7, especially up to and including maximally 4, carbon atoms, said moiety being branched (one or more times) or straight-chained. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Halo or halogen is preferably fluoro, chloro, bromo or iodo, most preferably fluoro, chloro or bromo.

Phenyl substituents Rb, Rc and Rd (if not hydrogen which is also preferred) are preferably
substituents —Y—B wherein Y is —C(═O)NR₅—, —NR₅C(═O)—, NR₅C(═O)NR₅—, —NR₅SO₂—, C(═O)—, —OC(═O)—, or —CO₂— and B is alkyl, substituted alkyl, alkenyl, substituted alkenyl, hydroxy, alkoxy, aryl, cycloalkyl, or when Y is —C(═O)NR₅—, B may also be —C(═O)R₆, —C(═O)R₆R₇ and —CO₂R₆, wherein R₅ is hydrogen or unsubstituted or substituted C₁-C₇ (or preferably C₁-C₄)-alkyl and R₆ and R₇ are, independently of each other, hydrogen, unsubstituted or substituted alkyl, unsubstituted or substituted aryl, cycloalkyl, unsubstituted or substituted heterocyclyl or, when attached to the same nitrogen atom, can form a heterocyclyl ring; where if at least one of the substituents Rb, Rc and Rd is —Y—B, then the corresponding compounds of the formula I are preferably for use in the treatment of a disease that depends on inadequate activity of a protein tyrosine kinase selected from c-Abl, c-Src and/or especially Ephrin receptor kinase, especially EphB4 kinase;
and/or one or more altered or mutated forms of any one or more of these (e.g. those that result in conversion of the respective proto-oncogene into an oncogene, such as constitutively activated Bcr-Abl or v-Src); and/or, preferably or, substituents selected from the group consisting of halo, especially fluoro, lower alkyl, substituted lower alkyl, such as halo lower alkyl, e.g. trifluoromethyl, lower alkenyl, lower alkynyl, phenyl, phenyl-lower alkyl, such as benzyl, lower alkanoyl, hydroxy, etherified or esterified hydroxy, such as lower alkoxy or lower alkanoyloxy, amino lower alkoxy, phenoxy or phenyl-lower alkoxy, such as benzyloxy, amino, mono- or disubstituted amino, wherein the substituents are selected from unsubstituted or substituted alkyl or from unsubstituted or substituted aryl, such as mono- or di-lower alkylamino, amidino, nitro, cyano, cyano-lower alkyl, carbamoyl, guanidino, ureido, unsubstituted or substituted mercapto, such as lower alkylthio, halogen-lower alkylthio, phenylthio, phenyl-lower alkylthio, lower alkyl-phenylthio, lower alkylsulfinyl, phenylsulfinyl, phenyl-lower alkylsulfinyl, lower alkylphenylsulfinyl, lower alkanesulfonyl, phenylsulfonyl, phenyl-lower alkylsulfonyl, lower alkylphenylsulfonyl, sulfonamido, or —NR_{X}R_{Y}, wherein R_{X} and R_{Y} together with the N atom form a 3- to 8-membered heterocyclic ring containing 1 to 4 nitrogen, oxygen or sulfur atoms (e.g. piperazino, lower alkyl-piperazino, azetidino, pyrrolidino, piperidino, morpholino, imidazolino),
or two of Ra, Rb and Rc together form a lower alkylene dioxy bridge bound at adjacent C-atoms of the phenyl ring, such as methylene or ethylene dioxy.

(“Unsubstituted”) alkyl preferably has 1 to 12 carbon atoms or is especially lower alkyl with up to 7 carbon atoms, preferably from 1 to and including 5, and is linear or branched; preferred is lower alkyl as defined above. In substituted alkyl, the alkyl (which is preferably as just defined) is substituted by one or more, preferably up to three, for example 1 or 2, substituents independently selected from phenyl that is unsubstituted or substituted, e.g. by halo, halo-lower alkyl, such as trifluoromethyl, amino, nitro or cyano; hydroxy-lower alkyl, such as hydroxymethyl, lower-alkoxy-lower alkyl, (lower-alkoxy)lower alkoxy-lower alkyl, lower alkanoyl-lower alkyl, phenoxy-lower alkyl, phenyl-lower alkoxy-lower alkyl, such as benzyloxy-lower alkyl, halo-lower alkyl, e.g. trifluoromethyl, lower alkenyl, lower alkynyl, lower alkanoyl, such as acetyl, lower alkoxy, hydroxy, lower alkoxy, phenoxy, phenyl-lower alkoxy, such as benzyloxy, amino, mono-di-substituted amino wherein the amino substituents are independently selected from lower alkyl, lower alkanoyl, phenyl and phenyl-lower alkyl; amino lower alkoxy; amidino, cyano, carboxy, lower alkoxy carbonyl, e.g. methoxy carbonyl, n-propoxy carbonyl or iso-propoxy carbonyl, phenyl-lower alkoxycarbonyl, such as benzyloxycarbonyl, lower alkanoyl, benzoyl, carbamoyl, N-mono- or N,N-disubstituted carbamoyl, such as N-mono- or N,N-di-lower alkylcarbamoyl or N-mono- or N,N-di-(hydroxy-lower alkyl)-carbamoyl, amidino, guanidino, ureido, mercapto, sulfo, lower alkylthio, sulfonamido, benzosulfonamido, phenyl, phenyl-lower alkyl, such as benzyl, phenoxy, phenyl-lower alkoxy, such as benzyloxy, phenylthio, phenyl-lower alkylthio, lower alkyl-phenylthio, lower alkylsulfinyl, phenylsulfinyl, phenyl-lower alkylsulfinyl, alkylphenylsulfinyl, lower alkanesulfonyl, phenylsulfonyl, phenyl-lower alkylsulfonyl, alkylphenylsulfonyl, halogen-lower alkylmercapto, halogen-lower alkylsulfonyl, such as trifluoromethane sulfonyl, or —NR_{X}R_{Y}, wherein Rₓ and R_{y} together with the N atom form a 3- to 8-membered heterocyclic ring further to one or more carbon ring atoms containing one to four nitrogen (where instead of an H in NH lower alkyl may be present), oxygen or sulfur atoms (e.g. azepino, diazepino (such as 1,4-diazepino), (especially N-) lower alkyl-diazepino, piperidino, morpholino, thiomorpholino, piperazino, (especially N-) lower alkyl-piperazino, pyrrolidino, imidazolidino, (especially N-) lower alkyl-imidazolidino, pyrazolidino, (especially N-) lower alkylpyrazolidino, azetidino or aziridino).

The term “3- to 8-membered” means having 3 to 8 ring atoms.

Alkenyl preferably has 2 to 12, more preferably 3 to 7, still more preferably 3 or 4 carbon atoms, e.g. in vinyl or allyl, and is (as far as chemically possible, as in some cases tautomerism or chemical instability e.g. in the case of substituents with active hydrogen adjacent to the double bond, e.g. with amino or hydroxy, may occur) substituted with one or more substituents independently selected from those mentioned as substituents for substituted alkyl.

Alkynyl preferably has 2 to 12, more preferably 3 to 7, still more preferably 3 or 4 carbon atoms, e.g. in vinyl or allyl, and is (as far as chemically possible, as in some cases tautomerism or chemical instability e.g. in the case of substituents with active hydrogen that are adjacent to the triple bond, e.g. with amino or hydroxy, may occur) substituted with one or more substituents independently selected from those mentioned as substituents for substituted alkyl.

In alkoxy, the alkyl moiety is preferably as defined above; preferred is lower alkoxy, such as methoxy or ethoxy.

Aryl is preferably an aromatic carbocyclic system of not more than 20 carbon atoms, especially not more than 16 carbon atoms, is preferably mono-, bi- or tri-cyclic, and is unsubstituted or, as substituted aryl, substituted preferably by one or more, preferably up to three, e.g. one or two substituents independently selected from those defined above under “substituted alkyl” and/or, in the case of aryl R₂, by a 3- to 8-membered heterocyclic ring, preferably bound via a ring nitrogen atom, containing, in addition to one or more carbon ring atoms, one to four nitrogen (where instead of an H in NH lower alkyl may be present), oxygen or sulfur atoms (e.g. azepinyl, diazepinyl (such as 1,4-diazepinyl), (especially N-) lower alkyl-diazepinyl, piperidinyl, morpholinyl, thiomorpholinyl, piperazinyl, (especially N-) lower alkyl-piperazinyl, pyrrolidinyl, imidazolidinyl, (especially N-) lower alkyl-imidazolidinyl, pyrazolidinyl, (especially N-) lower alkylpyrazolidinyl, azetidinyl or aziridinyl) which ring is unsubstituted or substituted (i) by a 3- to 8-membered heterocyclic ring, preferably bound via a ring nitrogen atom, containing, in addition to one or more carbon ring atoms, one to four nitrogen (where instead of an H in NH lower alkyl may be present), oxygen or sulfur atoms (e.g. azepinyl, diazepinyl (such as 1,4-diazepinyl), (especially N-) lower alkyl-diazepinyl, piperidinyl, morpholinyl, thiomorpholinyl, piperazinyl, (especially N-) lower alkyl-piperazinyl, pyrrolidinyl, imidazolidinyl, (especially N-) lower alkyl-imidazolidinyl, pyrazolidinyl, (especially N-) lower alkylpyrazolidinyl, azetidinyl or aziridinyl); (ii) by amino-lower alkyl or by N-mono or N,N-disubstituted amino-lower alkyl, wherein the amino substituents are preferably independently selected from lower alkyl, lower alkanoyl, phenyl and phenyl-lower alkyl, or (iii) by hydroxy-lower alkyl, e.g. hydroxymethyl, or etherified or esterified hydroxy-lower alkyl, e.g. lower-alkoxy-lower alkyl, (lower-alkoxy)-lower alkoxy-lower alkyl, lower alkanoyl-lower alkyl, phenoxy-lower alkyl, phenyl-lower alkoxy-lower alkyl, such as benzyloxy-lower alkyl, lower alkoxy-carbonyloxy-lower alkyl, such as tert-butoxycarbonyloxy-lower alkyl or phenyl-lower alkoxycarbonyloxy-lower alkyl, such as benzyloxycarbonyloxy-lower alkyl. For example, aryl is especially selected from phenyl, naphthyl, indenyl, azulenyl and anthryl, preferably phenyl, and is preferably in each case unsubstituted or substituted as just mentioned, especially by lower alkoxy or a 3- to 8-membered heterocyclic ring substituted by a 3- to 8-membered ring, by amino-lower alkyl, by N-mono- or N,N-di-substituted amino-lower alkyl, by hydroxy-lower alkyl or by esterified hydroxy-lower alkyl, in each case preferably as mentioned in this paragraph.

Cycloalkyl is preferably a saturated mono- or bicyclic hydrocarbon group with 3 to 9 ring carbon atoms, e.g. cyclopropyl, cyclobutyl, cyclopentyl, cyclohexyl, cycloheptyl or cyclooctyl.

In unsubstituted or substituted heterocyclyl, heterocyclyl is preferably a heterocyclic radical that is unsaturated, saturated or partially saturated in the bonding ring and is preferably a monocyclic or in a broader aspect of the invention bicyclic or tricyclic ring; has 3 to 24, more preferably 4 to 16 ring atoms; wherein at least in the ring bonding to the remaining part of the molecule of formula I one or more, preferably one to four, especially one or two carbon ring atoms are replaced by a heteroatom selected from the group consisting of nitrogen, oxygen and sulfur, the bonding ring preferably having 4 to 12, especially 5 to 7 ring atoms; heterocyclyl being unsubstituted or substituted by one or more, especially 1 to 3, substituents independently selected from the group consisting of the substituents defined above under “substituted alkyl” or “substituted aryl”; especially being a heterocyclyl radical selected from the group consisting of oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, pyranyol, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, beta-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, each of these radicals being unsubstituted or substituted by one to two radicals selected from the group consisting of lower alkyl, especially methyl or tert-butyl, lower alkoxy, especially methoxy, and halo, especially bromo or chloro.

Etherified or esterified hydroxy is preferably hydroxy etherified by unsubstituted or substituted lower alkyl which is preferably as defined above, and is more preferably lower-alkoxy, (lower-alkoxy)-lower alkoxy, phenoxy, phenyl-lower alkoxy, such as benzyloxy, or hydroxy esterified by an organic carbonic or sulfonic acid, e.g. lower alkanoyloxy, lower alkoxy-carbonyloxy, such as tert-butoxycarbonyloxy, phenyl-lower alkoxy-carbonyloxy, such as benzyloxycarbonyloxy, methylphenylsulfonyloxy or lower-alkylsulfonyloxy.

In mono- or disubstituted amino, one or both of the hydrogen atoms of an amino group —NH₂ are replaced by a substituent, preferably (if not indicated specifically otherwise) independently selected from unsubstituted or substituted alkyl wherein in case of substituted alkyl the substitutents are independently selected from those mentioned under “substituted alkyl”, from unsubstituted or substituted aryl wherein the substituents are as defined under “substituted aryl”, preferably as defined under “substituted alkyl”, and from unsubstituted or substituted lower alkanoyl wherein in case of substituted alkanoyl the substitutents are independently selected from those mentioned under “substituted alkyl”, such as lower-alkanoylamino; preferably, in mono- or disubstituted amino the substituents are independently selected from lower alkanoyl or more preferably from lower alkyl and phenyl-lower alkyl, e.g. in mono- or di-lower alkylamino or mono- or di-(phenyl-lower alkyl)-amino.

Lower alkanoyl preferably is the acyl moiety of a carbonic acid with up to seven, more preferably with up to 4 carbon atoms, and is, for example, formyl or preferably acetyl, propionyl or butyroyl.

In substituted mercapto, the mercapto hydrogen is either substituted by unsubstituted or substituted lower alkyl which is preferably as defined above, and is more preferably lower-alkylthio, (lower-alkoxy)-lower alkylthio, phenylthio, phenyl-lower alkylthio, such as benzylthio; or by an organic carbonic acid, e.g. in lower alkanoylthio, lower alkoxy-carbonylthio, such as tert-butoxycarbonylthio, phenyl-lower alkoxycarbonylthio, such as benzyloxycarbonyl-thio.

Unsubstituted or substituted aryl R₂ is preferably monocyclic aryl, more preferably phenyl, that is unsubstituted or especially substituted (especially in m- or p-position) by
a substituent which is a 3- to 8-membered heterocyclic ring, preferably bound via a ring nitrogen atom, containing, in addition to one or more carbon ring atoms, one to four nitrogen (where instead of an H in NH lower alkyl may be present), oxygen or sulfur atoms (e.g. azepinyl, diazepinyl (such as 1,4-diazepinyl), (especially N-) lower alkyl-diazepinyl, piperidinyl, morpholinyl, thiomorpholinyl, piperazinyl, (especially N-) lower alkyl-piperazinyl, pyrrolidinyl, imidazolidinyl, (especially N-) lower alkyl-imidazolidinyl, pyrazolidinyl, (especially N-) lower alkylpyrazolidinyl, azetidinyl or aziridinyl) which ring is unsubstituted or substituted by either a 3- to 8-membered heterocyclic ring, preferably bound via a ring carbon or nitrogen atom, containing, in addition to one or more carbon ring atoms, one to four nitrogen (where instead of an H in NH lower alkyl may be present), oxygen or sulfur atoms (e.g. azepinyl, diazepinyl (such as 1,4-diazepinyl), (especially N-) lower alkyl-diazepinyl, piperidinyl, morpholinyl, thiomorpholinyl, piperazinyl, (especially N-) lower alkyl-piperazinyl, pyrrolidinyl, imidazolidinyl, (especially N-) lower alkyl-imidazolidinyl, pyrazolidinyl, (especially N-) lower alkylpyrazolidinyl, azetidinyl or aziridinyl or
by amino-lower alkyl or by N-mono or N,N-disubstituted amino-lower alkyl, wherein the amino substituents are preferably independently selected from lower alkyl, lower alkanoyl, phenyl and phenyl-lower alkyl, e.g. N,N-di-(lower alkyl)amino-lower alkyl, such as N,N-dimethylamino-lower alkyl, or
by hydroxy-lower alkyl, e.g. hydroxymethyl, or etherified or esterified hydroxy-lower alkyl, e.g. lower-alkoxy-lower alkyl, (lower-alkoxy)-lower alkoxy-lower alkyl, lower alkanoyl-lower alkyl, phenoxy-lower alkyl, phenyl-lower alkoxy-lower alkyl, such as benzyloxy-lower alkyl, lower alkoxy-carbonyloxy-lower alkyl, such as tert-butoxycarbonyloxy-lower alkyl or phenyl-lower alkoxycarbonyloxy-lower alkyl, such as benzyloxycarbonyloxy-lower alkyl; or,
in a more general aspect of the invention, by lower alkoxy, such as methoxy.

Unsubstituted or substituted alkyl R₃ is as defined for unsubstituted or substituted alkyl above; preferred is unsubstituted or substituted lower alkyl, especially lower alkyl, such as methyl or ethyl, or mono- or disubstituted amino-lower alkyl, wherein lower alkyl is preferably methyl, ethyl, propyl or butyl, more preferably substituted at the terminal carbon atom (the one most removed from the ring in formula I) by unsubstituted or preferably mono- or disubstituted amino wherein mono- or disubstituted amino is as defined above, preferably mono- or di-lower alkylamino, such as N,N-dimethylamino or N,N-diethylamino, for example in 3-(N,N-dimethylamino)-propyl.

R₄ is preferably hydrogen.

Salts are especially the pharmaceutically acceptable salts of compounds of formula I. They can be formed where salt forming groups, such as basic or acidic groups, are pre-sent that can exist in dissociated form at least partially, e.g. in a pH range from 4 to 10 in aqueous solutions, or can be isolated especially in solid form.

Such salts are formed, for example, as acid addition salts, preferably with organic or inorganic acids, from compounds of formula I with a basic nitrogen atom (e.g. in an imino or amino group), especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable comprised in pharmaceutical preparations), and these are therefore preferred.

In view of the close relationship between the compounds in free form and in the form of their salts, including those salts that can be used as intermediates, for example in the purification or identification of the compounds or salts thereof, any reference to “compounds” and “intermediates” hereinbefore and hereinafter, especially to the compound(s) of the formula I, is to be understood as referring also to one or more salts thereof or a mixture of a free compound and one or more salts thereof, each of which is intended to include also any solvate, metabolic precursor such as ester or amide of the compound of formula I, or salt of any one or more of these, as appropriate and expedient and if not explicitly mentioned otherwise. Different crystal forms may be obtainable and then are also included.

Where the plural form is used for compounds, salts, pharmaceutical preparations, diseases, disorders and the like, this is intended to mean one (preferred) or more single compound(s), salt(s), pharmaceutical preparation(s), disease(s), disorder(s) or the like, where the singular or the indefinite article (“a”, “an”) is used, this is intended to include the plural or preferably the singular.

In some cases, a compound of the present invention comprises one or more chiral centers or show other asymmetry (leading to enantiomers) or may otherwise be able to exist in the form of more than one stereoisomer, e.g. due more than one chiral centers or more than one asymmetry or due to rings or double bonds that allow for Z/E (or cis-trans) isomerism (diastereomers). The present inventions includes both mixtures of two or more such isomers, such as mixtures of enantiomers, especially racemates, as well as preferably purified isomers, especially purified enantiomers or enantiomerically enriched mixtures.

The compounds of formula I have valuable pharmacological properties and are useful in the treatment of protein kinase dependent diseases or disorder, especially diseases or disorder dependent on inadequate expression of a protein tyrosine kinase, preferably one or more of those mentioned above as preferred, e.g., as drugs or as basis for pharmaceutical formulations to treat one or more proliferative diseases depending on inadequate activity of a protein tyrosine kinase, especially one or more of the preferred ones just mentioned.

The terms “treatment” or “therapy” refer to the prophylactic (e.g. delaying or preventing the onset of a disease or disorder) or preferably therapeutic (including but not limited to palliative, curing, symptom-alleviating, symptom-reducing, patient condition ameliorating, kinase-regulating and/or kinase-inhibiting) treatment of said disease(s) or disorder(s), especially of the one or more disease or disorder mentioned above or below.

A warm-blooded animal (or patient) is preferably a mammal, especially a human.

“Inadequate” kinase activity preferably relates to a state of a warm-blooded animal where a kinase, especially one mentioned above or below, shows a kinase activity that is too high in the given situation (e.g. due to one or more of deregulation, overexpression e.g. due to gene amplification or chromosome rearrangement or infection by microorganisms such as virus that express an aberrant gene, e.g. an oncogene, abnormal activity e.g. leading to an erroneous substrate specificity or a hyperactive protein e.g. produced in normal amounts, and/or the like) and/or leads to or supports a kinase dependent disease or disorder as mentioned above and below, e.g. by modification (such as phosphorylation, cleavage or the like) of the kinase leading to inadequate kinase activity. Such inadequate kinase activity may, for example, comprise a higher than normal activity, or further an activity in the normal or even below the normal range which, however, due to preceding, parallel and or subsequent processes, e.g. signaling, regulatory effect on other processes and the like, leads to direct or indirect support or maintenance of a disease or disorder, and/or an activity that supports the outbreak and/or presence of a disease or disorder in any other way. The inadequate activity of the relevant protein kinases, especially protein tyrosine kinases, may or may not be dependent on parallel other mechanisms supporting the disorder or disease, and/or the prophylactic or therapeutic effect may or may include other mechanisms in addition to inhibition of a protein kinase, especially a protein tyrosine kinase, especially one of those mentioned as being preferred which are the preferred targets for inhibition. Therefore “dependent” has to be read as “dependent inter alia”, (especially in cases where a disease or disorder is really exclusively dependent only on one protein kinase, preferably a protein tyrosine kinase) preferably as “dependent mainly”, more preferably as “dependent essentially only”.

Where a disease or disorder dependent on inadequate activity of a protein kinase, especially a protein tyrosine kinase, is mentioned (such in the definition of “use” in the following paragraph and also especially where a compound of the formula I is mentioned for use in the diagnostic or therapeutic treatment which is preferably the treatment of a disease or disorder dependent on inadequate activity of a protein (preferably tyrosine) kinase), this refers preferably to any one or more diseases or disorders that depend on inadequate activity one or more of c-Abl, c-Src and/or especially Ephrin receptor kinase, especially EphB4 kinase; and/or one or more altered or mutated forms of any one or more of these (e.g. those that result in conversion of the respective proto-oncogene into an oncogene, such as constitutively activated Bcr-Abl or v-Src).

Where subsequently or above the term “use” is mentioned (as verb or noun) (relating to the use of a compound of the formula I or a pharmaceutically acceptable salt thereof), this (if not indicated differently in the context) includes any one or more of the following embodiments of the invention, respectively (if not stated otherwise): the use in the treatment of a disease or disorder that depends on inadequate activity of a protein (preferably tyrosine) kinase, the use for the manufacture of pharmaceutical compositions for use in the treatment of a disease or disorder that depends on inadequate activity of a protein (preferably tyrosine) kinase; a method of use of one or more compounds of the formula I in the treatment of a disease or disorder that depends on inadequate activity of a protein (preferably tyrosine) kinase; a pharmaceutical preparation comprising one or more compounds of the formula I for the treatment of a disease or disorder that depends on inadequate activity of a protein (preferably tyrosine) kinase; and one or more compounds of the formula I for use in the treatment of a disease or disorder that depends on inadequate activity of a protein (preferably tyrosine) kinase, especially any one or more of the protein tyrosine kinases mentioned as preferred; as appropriate and expedient, if not stated otherwise.

The compounds of formula I have valuable pharmacological properties and can be used in the treatment of protein kinase, especially protein tyrosine kinase, dependent diseases, e.g., as drugs to treat proliferative diseases.

In the following description of typical exemplary testing systems, the following abbreviations have the following meanings: DMSO=dimethyl sulfoxide; DTT=dithiothreitol; EDTA=ethylene diamine tetraacetate; MOI=multiplicity of infection; PMSF=p-toluenesulfonyl fluoride; Tris=tris(hydroxymethyl)aminomethane. An “inhibitor” is a test compound of the formula I if not mentioned otherwise.

The (especially important and preferred) efficacy of compounds of the formula I as inhibitors or Ephrin B4 receptor (EphB4) kinases can be demonstrated as follows:

Generation of Bac-to-Bac™ (Invitrogen Life Technologies, Basel, Switzerland) GST-fusion expression vectors: Entire cytoplasmatic coding regions of the EphB-class are amplified by PCR from cDNA libraries derived from human placenta or brain, respectively. Recombinant baculovirus are generated that express the amino acid region 566-987 of the human EphB4 receptor (SwissProt Database, Accession No. P54760). GST sequence is cloned into pFastBac1® vector (Invitrogen Life Technologies, Basel, Switzerland) and PCR amplified. cDNAs encoding EphB4-receptor domains, respectively are cloned in frame 3′prime to the GST sequence into this modified FastBac1 vector to generate pBac-to-Bac™ donor vectors. Single colonies arising from the transformation are inoculated to give overnight cultures for small scale plasmid preparation. Restriction enzyme analysis of plasmid DNA reveals several clones to contain inserts of the expected size. By automated sequencing the inserts and approximately 50 bp of the flanking vector sequences are confirmed on both strands.

Production of viruses: Viruses for each of the kinases are made according to the protocol supplied by GIBCO if not stated otherwise. In brief, transfer vectors containing the kinase domains are transfected into the DH10Bac cell line (GIBCO) and plated on selective agar plates. Colonies without insertion of the fusion sequence into the viral genome (carried by the bacteria) are blue. Single white colonies are picked and viral DNA (bac-mid) isolated from the bacteria by standard plasmid purification procedures. Sf9 cells or Sf21 cells are then transfected in 25 cm² flasks with the viral DNA using Cellfectin reagent according to the protocol.

Purification of GST-tagged kinases: The centrifuged cell lysate is loaded onto a 2 mL glutathione-sepharose column (Pharmacia) and washed three times with 10 mL of 25 mM Tris-HCl, pH 7.5, 2 mM EDTA, 1 mM DTT, 200 mM NaCl. The GST-tagged proteins are then eluted by 10 applications (1 mL each) of 25 mM Tris-HCl, pH 7.5, 10 mM reduced-glutathione, 100 mM NaCl, 1 mM DTT, 10% Glycerol and stored at −70° C.

Protein kinase assays: The activities of protein kinases are assayed in the presence or absence of inhibitors, by measuring the incorporation of ³³P from [γ³³P]ATP into a polymer of glutamic acid and tyrosine (poly(Glu,Tyr)) as a substrate. The kinase assays with purified GST-EphB (30 ng) are carried out for 15-30 min at ambient temperature in a final volume of 30 μL containing 20 mM Tris.HCl, pH 7.5, 10 mM MgCl₂, 3-50 mM MnCl₂, 0.01 mM Na₃VO₄, 1% DMSO, 1 mM DTT, 3 μg/mL poly(Glu,Tyr) 4:1 (Sigma; St. Louis, Mo., USA) and 2.0-3.0 μM ATP (γ-[³³P]-ATP 0.1 μCi). The assay is terminated by the addition of 20 μL of 125 mM EDTA. Subsequently, 40 μl of the reaction mixture are transferred onto Immobilon-PVDF membrane (Millipore, Bedford, Mass., USA) previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μl 0.5% H₃PO₄. Membranes are removed and washed 4× on a shaker with 1.0% H₃PO₄, once with ethanol. Membranes are counted after drying at ambient temperature, mounting in Packard TopCount96-well frame, and addition of 10 μL/well of Microscint™ (Packard). IC₅₀ values are calculated by linear regression analysis of the percentage inhibition of each compound in duplicate, at four concentrations (usually 0.01, 0.1, 1 and 10 μM). One unit of protein kinase activity is defined as 1 nmole of ³³P ATP transferred from [γ³³P] ATP to the substrate protein per minute per mg of protein at 37° C. Compounds of formula I show EphB4 inhibition down to 1 nM, preferably IC₅₀ values between 0.001-5.0 μM.

The efficacy of the compounds of the invention as inhibitors of c-Abl protein-tyrosine kinase activity can be demonstrated as follows:

An in vitro enzyme assay is performed in 96-well plates as a filter binding assay as described by Geissler et al. in Cancer Res. 1992; 52:4492-4498, with the following modifications. The His-tagged kinase domain of c-Abl is cloned and expressed in the baculovirus/Sf9 system as described by Bhat et al. in J. Biol. Chem. 1997; 272:16170-16175. A protein of 37 kD (c-Abl kinase) is purified by a two-step procedure over a Cobalt metal chelate column followed by an anion exchange column with a yield of 1-2 mg/L of Sf9 cells (Bhat et al., reference cited). The purity of the c-Abl kinase is >90% as judged by SDS-PAGE after Coomassie blue staining. The assay contains (total volume of 30 μL): c-Abl kinase (50 ng), 20 mM Tris.HCl, pH 7.5, 10 mM MgCl₂, 10 μM Na₃VO₄, 1 mM DTT and 0.06 μCi/assay [γ³³ P]-ATP (5 μM ATP) using 30 μg/mL poly-Ala,Glu,Lys,Tyr-6:2:5:1 (Poly-AEKY, Sigma P1152) in the presence of 1% DMSO. Reactions are terminated by adding 10 μL of 250 mM EDTA and 30 μL of the reaction mixture is transferred onto Immobilon-PVDF membrane (Millipore, Bedford, Mass., USA) previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μL 0.5% H₃PO₄. Membranes are removed and washed on a shaker with 0.5% H₃PO₄ (4 times) and once with ethanol. Membranes are counted after drying at ambient temperature, mounting in Packard TopCount 96-well frame, and addition of 10 μL/well of Microscint™ (Packard). Using this test system, compounds of the formula I can show IC₅₀ values of inhibition for c-Abl inhibition in the range of e.g. 0.002 to 100 μM, usually between 0.002 and 5 μM.

Alternatively, EphB4 receptor autophosphorylation can be measured as follows:

The inhibition of EphB4 receptor autophosphorylation can be confirmed with an in vitro experiment in cells such as transfected A375 human melanoma cells (ATCC Number: CRL-1619), which permanently express human EphB4 (SwissProt AccNo P54760), are seeded in complete culture medium (with 10% fetal calf serum=FCS) in 6-well cell-culture plates and incubated at 37° C. under 5% CO2 until they show about 90% confluency. The compounds to be tested are then diluted in culture medium (without FCS, with 0.1% bovine serum albumin) and added to the cells. (Controls comprise medium without test compounds). Ligand induced autophosphorylation is induced by the addition of 1 microg/ml soluble ephrinB2-Fc (s-ephrinB2-Fc: R&D Biosystems, CatNr 496-EB) and 0.1 microM ortho-vanadate. After a further 20 minutes incubation at 37° C., the cells are washed twice with ice-cold PBS (phosphate-buffered saline) and immediately lysed in 200 μl lysis buffer per well. The lysates are then centrifuged to remove the cell nuclei, and the protein concentrations of the supernatants are determined using a commercial protein assay (PIERCE). The lysates can then either be immediately used or, if necessary, stored at −20° C.

A sandwich ELISA is carried out to measure the EphB4 phosphorylation: To capture phosphorylated EphB4 protein 100 ng/well of ephrinB2-Fc (s-ephrinB2-Fc: R&D Biosystems, CatNr 496-EB) is immobilized MaxiSorb (Nunc) ELISA plates. The plates are then washed and the remaining free protein-binding sites are saturated with 3% TopBlock® (Juro, Cat. # TB232010) in phosphate buffered saline with Tween 20® (polyoxyethylen(20)sorbitane monolaurate, ICI/Uniquema) (PBST). The cell lysates (100 μg protein per well) are then incubated in these plates for 1 h at room temperature. After washing the wells three times with PBS an antiphosphotyrosine antibody coupled with alkaline phosphatase (PY 20 Alkaline Phosphate conjugated: ZYMED, Cat Nr03-7722) is added and incubated for another hour. The plates are washed again and the binding of the antiphosphotyrosine antibody to the captured phosphorylated receptor is then demonstrated and quantified using 10 mM D-nitrophenylphosphat as substrate and measuring the OD at 405 nm after 0.5 h-1 h.

The difference between the signal of the positive control (stimulated with vanadate and s-ephrinB2-Fc) and that of the negative control (not stimulated) corresponds to maximal EphB4 phosphorylation (=100%). The activity of the tested substances is calculated as percent inhibition of maximal EphB4 phosphorylation, wherein the concentration of substance that induces half the maximum inhibition is defined as the IC₅₀ (inhibitory dose for 50% inhibition).

The compounds of formula I can also inhibit other tyrosine protein kinases such as especially the c-Src kinase which plays a part in growth regulation and transformation in animals, especially mammal cells, including human cells. An appropriate assay is described in Andrejauskas-Buchdunger et al., Cancer Res. 52, 5353-8 (1992). Using this test system, compounds of the formula I can show IC₅₀ values for inhibition of c-Src in the range of e.g. 0.01 to 100 μM, usually between 0.1 and 10 μM.

On the other hand, the compounds of the formula I show preferably rather low inhibition of various other protein tyrosine or serine/threonine kinases and thus display a useful selectivity with a diminished risk of undesired adverse drug reactions.

For example, the activity of the compounds of the invention as inhibitors of KDR protein-tyrosine kinase activity can be demonstrated as follows: The inhibition of VEGF-induced receptor autophosphorylation can be confirmed in cells such as transfected CHO cells, which permanently express human VEGF-R2 receptor (KDR), and are seeded in complete culture medium (with 10% fetal calf serum=FCS) in 6-well cell-culture plates and incubated at 37° C. under 5% CO₂ until they show about 80% confluency. The compounds to be tested are then diluted in culture medium (without FCS, with 0.1% bovine serum albumin) and added to the cells. Controls comprise medium without test compounds. After 2 h incubation at 37° C., recombinant VEGF is added; the final VEGF concentration is 20 ng/ml. After a further incubation period of five minutes at 37° C., the cells are washed twice with ice-cold PBS (phosphate-buffered saline) and immediately lysed in 100 μl lysis buffer per well. The lysates are then centrifuged to remove the cell nuclei, and the protein concentrations of the supernatants are determined using a commercial protein assay (BIORAD). The lysates can then either be immediately used or, if necessary, stored at −20° C. Using this protocol, selective compounds of the formula I can be found to show IC₅₀ values for KDR inhibition that are preferably at least 4 times higher than for EphB4 tyrosine kinase, more preferably more than 20 times higher than for EphB4 tyrosine kinase.

The relatively low inhibition of Tek can be determined as follows: The procedure of the expression, purification and assay for this kinase has been described. Fabbro et al., Pharmacol. Ther. 82(2-3) 293-301 (1999). Selective compounds of formula I can show IC₅₀ values, calculated by linear regression analysis, for Tek inhibition that are preferably at least 4 times, more preferably more than 20 times higher than for EphB4 inhibition.

The results indicate an advantageous selectivity profile of preferred compounds of the formula I, where selectivity does not necessarily mean that only one kinase is inhibited to an advantageous extent, but also that selectively two or more kinases may be inhibited stronger in comparison to other kinases.

There are also experiments to demonstrate the antitumor activity of compounds of the formula I in vivo. For example, in order to test whether a compound of the formula I, e.g. that of Example 1 given below, inhibits angiogenesis in vivo, its effect on the angiogenic response induced by an angiogenenic factor such as VEGF, bFGF, S-1P. PDGF or IGF-1 in a growth factor implant model in mice is tested: A porous Teflon chamber (volume 0.5 mL) is filled with 0.8% w/v agar containing heparin (20 units/ml) with or without growth factor (2 μg/ml human VEGF) is implanted subcutaneously on the dorsal flank of C57/C6 mice. The mice are treated with the test compound (e.g. 5, 10, 25, 50 or 100 mg/kg p.o. once daily) or vehicle starting on the day of implantation of the chamber and continuing for 4 days after. At the end of the treatment, the mice are killed, and the chambers are removed. The vascularized tissue growing around the chamber is carefully removed and weighed, and the blood content is assessed by measuring the hemoglobin content of the tissue (Drabkins method; Sigma, Deisenhofen, Germany). Tie-2 protein levels, as a measure of an endothelial marker, are determined by a specific ELISA to quantify the angiogenic response. It has been shown previously that these growth factors induce dose-dependent increases in weight, blood content and Tie-2 protein levels of this tissue growing (characterized histologically to contain fibroblasts and small blood vessels) around the chambers and that this response is blocked by neutralizing antibodies e.g. that specifically neutralize VEGF (see Wood J M et al., Cancer Res. 60(8), 2178-2189, (2000); and Schlaeppi et al., J. Cancer Res. Clin. Oncol. 125, 336-342, (1999)). With this model, inhibition can be shown in the case of compounds of the formula I at the concentrations given above.

In a preferred sense of the invention, a disease or disorder dependent on inadequate activity of a protein (preferably tyrosine) kinase, especially one characterized as being preferred above, where a compound of the formula I can be used is one or more of a proliferative disease (meaning one dependent on inadequate including a hyperproliferative condition, such as one or more of leukemia, hyperplasia, fibrosis (especially pulmonary, but also other types of fibrosis, such as renal fibrosis), angiogenesis, psoriasis, atherosclerosis and smooth muscle proliferation in the blood vessels, such as stepnosis or restenosis following angioplasty. Further, a compound of the formula I may be used for the treatment of thrombosis and/or scleroderma.

Preferred is the use of a compound of the formula I in the therapy (including prophylaxis) of a proliferative disorder (especially which is dependent on inadequate activity of a protein (preferably tyrosine) kinase, especially as mentioned as preferred herein) selected from tumor or cancer diseases, especially against preferably a benign or especially malignant tumor or cancer disease, more preferably solid tumors, e.g. carcinoma of the brain, kidney, liver, adrenal gland, bladder, breast, stomach (especially gastric tumors), ovaries, colon, rectum, prostate, pancreas, lung (e.g. small or large cell lung carcinomas), vagina, thyroid, sarcoma, glioblastomas, multiple myeloma or gastrointestinal cancer, especially colon carcinoma or colorectal adenoma, or a tumor of the neck and head, e.g. squameous carcinoma of the head and neck, including neoplasias, especially of epithelial character, e.g. in the case of mammary carcinoma; an epidermal hyperproliferation (other than cancer), especially psoriasis; prostate hyperplasia; or a leukemia.

A compound of formula I or its use makes it possible to bring about the regression of tumors and to prevent the formation of tumor metastases and the growth of (also micro)metastases. It is also possible to use the compounds of formula I in the treatment of diseases of the immune system insofar as several or, especially, individual protein (preferably tyrosine) kinases, especially those mentioned as preferred, are involved; furthermore, the compounds of formula I can be used also in the treatment of diseases of the central or peripheral nervous system where signal transmission by at least one protein (preferably tyrosine) kinase, especially selected from those protein tyrosine kinases mentioned as preferred, is involved.

In chronic myelogenous leukemia (CML), a reciprocally balanced chromosomal translocation in hematopoietic stem cells (HSCs) produces the BCR-ABL hybrid gene. The latter encodes the oncogenic Bcr-Abl fusion protein. Whereas ABL encodes a tightly regulated protein tyrosine kinase, which plays a fundamental role in regulating cell proliferation, adherence and apoptosis, the BCR-ABL fusion gene encodes as constitutively activated kinase which transforms HSCs to produce a phenotype exhibiting deregulated clonal proliferation, reduced capacity to adhere to the bone marrow stroma and a reduced apoptotic response to mutagenic stimuli, which enable it to accumulate progresssively more malignant transformations. The resulting granulocytes fail to develop into mature lymphocytes and are released into the circulation, leading to a deficiency in the mature cells and increased infection susceptibility. ATP-competitive inhibitors of Bcr-Abl have been described that prevent the kinase from activating mitogenic and anti-apoptotic pathways (e.g. P-3 kinase and STAT5), leading to the death of the BCR-ABL phenotype cells and thus providing an effective therapy against CML. The 3,4-substituted pyrazolopyrimidin-derivatives useful according to the present invention, especially the compounds of formula I, as Bcr-Abl inhibitors are thus especially appropriate for the therapy of diseases related to its overexpression, especially leukemias, such as leukemias, e.g. CML or ALL.

Angiogenesis is regarded as an absolute prerequisite for those tumors which grow beyond a maximum diameter of about 1-2 mm; up to this limit, oxygen and nutrients may be supplied to the tumor cells by diffusion. Every tumor, regardless of its origin and its cause, is thus dependent on angiogenesis for its growth after it has reached a certain size. Three principal mechanisms play an important role in the activity of angiogenesis inhibitors against tumors: 1) Inhibition of the growth of vessels, especially capillaries, into avascular resting tumors, with the result that there is no net tumor growth owing to the balance that is achieved between apoptosis and proliferation; 2) Prevention of the migration of tumor cells owing to the absence of blood flow to and from tumors; and 3) Inhibition of endothelial cell proliferation, thus avoiding the paracrine growth-stimulating effect exerted on the surrounding tissue by the endothelial cells normally lining the vessels.

Compounds of the formula I, in regard of their ability to inhibit KDR and Ephrin receptor kinase, especially EphB4 kinase, and possibly other protein kinases, and thus to modulate angiogenesis, are especially appropriate for the use against diseases or disorders related to the inadequate activity of the corresponding receptor (preferably tyrosine) kinase, especially an overexpression thereof. Among these diseases, especially (e.g. ischemic) retinopathies, (e.g. age related) macula degeneration, psoriasis, obesity, haemangioblastoma, haemangioma, inflammatory diseases, such as rheumatoid or rheumatic inflammatory diseases, especially arthritis, such as rheumatoid arthritis, or other chronic inflammatory disorders, such as chronic asthma, arterial or post-transplantational atherosclerosis, endometriosis, and especially neoplastic diseases, for example so-called solid tumors (especially cancers of the gastrointestinal tract, the pancreas, breast, stomach, cervix, bladder, kidney, prostate, ovaries, endometrium, lung, brain, melanoma, Kaposi's sarcoma, squamous cell carcinoma of head and neck, malignant pleural mesotherioma, lymphoma or multiple myeloma) and further liquid tumors (e.g. leukemias) are especially important.

The compounds of the formula I are especially of use to prevent or treat diseases that are triggered by persistent angiogenesis, such as restenosis, e.g., stent-induced restenosis; Crohn's disease; Hodgkin's disease; eye diseases, such as diabetic retinopathy and neovascular glaucoma; renal diseases, such as glomerulonephritis; diabetic nephropathy; inflammatory bowel disease; malignant nephroscierosis; thrombotic microangiopathic syndromes; (e.g. chronic) transplant rejections and glomerulopathy; fibrotic diseases, such as cirrhosis of the liver; mesangial cell-proliferative diseases; injuries of the nerve tissue; and for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

Preferably, the invention relates to the use of compounds of the formula I, or pharmaceutically acceptable salts thereof, in the treatment of solid tumors as mentioned herein and/or of liquid tumors, e.g. leukemias, as mentioned herein.

Process of Manufacture

A compound of formula I is prepared analogously to methods that, for other compounds, are in principle known in the art, so that for the novel compounds of the formula I the process is novel as analogy process, preferably by reacting a pyrazolopyrimidine compound of the formula II, 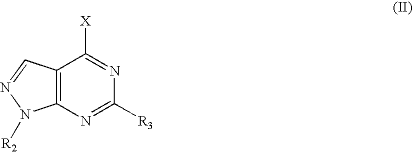
wherein R₂ and R₃ are as defined for a compound of the formula I and X is hydroxy or a leaving group (especially halo), with an amino compound of the formula III,
H—NR₄—R₁ (III)
wherein R₁ and R₄ are as defined for a compound of the formula I; and, if desired, transforming a compound of formula I into a different compound of formula I, transforming a salt of an obtainable compound of formula I into the free compound or a different salt, transforming an obtainable free compound of formula I into a salt thereof, and/or separating an obtainable mixture of isomers of a compound of formula I into individual isomers.

The reaction takes place under conditions that, as such, are known in the art, preferably in an appropriate solvent, e.g. a N,N-di-lower alkyl-lower alkanoylamide, such as N,N-dimethyl formamide, or an alcohol, e.g. a hydroxy-lower alkane, such as methanol or ethanol, preferably at temperatures between 15° C. and 160° C., e.g. between room temperature and 150° C. or under reflux. The reaction preferably takes place under an inert gas, such as nitrogen or argon, and preferably the solvents are free of water, especially absolute solvents.

A leaving group X in a compound of the formula III is preferably halo, preferably iodo, bromo or especially chloro, methylphenylsulfonyloxy, such as toluoyloxy, or perfluoroalkyl-sulfonyloxy (e.g. —O—SO₂—(C_{f}F_{2f+1}), wherein f=1, 2 or 4).

Optional Reactions and Conversions

Compounds of the formula I may be converted into different compounds of the formula I. For example, in a compound where R₂ is halophenyl, especially 4-bromophenyl, may be converted into the corresponding compound wherein R₂ is phenyl is (especially 4-) substituted by a 3- to 8-membered heterocyclic ring further to one or more carbon ring atoms containing one to four nitrogen (where instead of an H in NH lower alkyl may be present), oxygen or sulfur atoms (e.g. azepino, diazepino (such as 1,4-diazepino), (especially N-) lower alkyl-diazepino, piperidino, morpholino, thiomorpholino, piperazino, (especially N-) lower alkyl-piperazino, pyrrolidino, imidazolidino, (especially N-) lower alkyl-imidazolidino, pyrazolidino, (especially N-) lower alkylpyrazolidino, azetidino or aziridino) which ring is unsubstituted or substituted by (i) by a 3- to 8-membered heterocyclic ring, preferably bound via a ring carbon or nitrogen atom, containing, in addition to one or more carbon ring atoms, one to four nitrogen (where instead of an H in NH lower alkyl may be present), oxygen or sulfur atoms, especially as defined above; (ii) by amino-lower alkyl or by N-mono or N,N-disubstituted amino-lower alkyl, preferably as defined above; or (iii) by hydroxy-lower alkyl or etherified or esterified hydroxy-lower alkyl, especially as defined above. The reaction preferably takes place under customary coupling conditions incubating the starting compound with R₂=halophenyl and the complementary heterocyclic ring introducing compound which has an —NH— which contains the nitrogen to bind instead of the halo moiety with a strong base, such as an alkali metal alcoholate, e.g. potassium tert-butoxide, in an appropriate solvent, such as an ether, e.g. tetrahydrofurane, in the presence of a catalyst, especially a complex palladium catalyst, such as 2-(dimethylamino)ferrocen-1-yl-palladium(II)chloride complex, preferably at elevated temperatures, e.g. from 50° C. to the reflux temperature, for example at 105 to 115° C.

Salts of compounds of formula I having at least one salt-forming group may be prepared in a manner known per se. For example, salts of compounds of formula I having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula I are obtained in customary manner, e.g. by treating the compounds with an acid or a suitable anion exchange reagent. Internal salts of compounds of formula I containing acid and basic salt-forming groups, e.g. a free carboxy group and a free amino group, may be formed, e.g. by the neutralization of salts, such as acid addition salts, to the isoelectric point, e.g. with weak bases, or by treatment with ion exchangers.

A salt of a compound of the formula I can be converted in customary manner into the free compound; metal and ammonium salts can be converted, for example, by treatment with suitable acids, and acid addition salts, for example, by treatment with a suitable basic agent. In both cases, suitable ion exchangers may be used.

Stereoisomeric mixtures, e.g. mixtures of diastereomers, can be separated into their corresponding isomers in a manner known per se by means of appropriate separation methods. Diastereomeric mixtures for example may be separated into their individual diastereomers by means of fractionated crystallization, chromatography, solvent distribution, and similar procedures. This separation may take place either at the level of one of the starting compounds or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

Intermediates and final products can be worked up and/or purified according to standard methods, e.g. using chromatographic methods, distribution methods, (re-) crystallization, and the like.

Starting Materials

The starting materials can, for example, preferably be prepared as follows: A pyrazolopyrimidine compound of the formula II is preferably prepared from a 4-hydroxy-pyrazolopyrimidine of the formula IV, 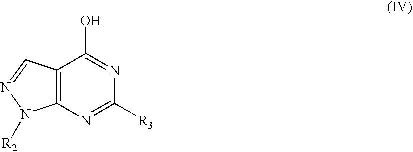
wherein R₂ and R₃ are as defined for a compound of the formula I, wherein the moiety —C(—OH)—N— may be in equilibrium with the tautomeric form —C(═O)—NH— or one of these two tautomeric forms may strongly prevail, with an anhydride of a methylphenylsulfonic acid or a perfluoroalkanesulfonic acid, e.g. the corresponding sulfonyl chloride or bromide, or preferably an acid halide such as phosgene, oxaloylchloride, more preferably an inorganic acid halide, such as thionyl chloride, thionyl bromide, sulfuryl chloride, phosphorus trichloride, phosphorus tribromide, phosphorus pentachloride, phosphorus pentabromide, phosphoryl bromide or especially phosphoryl chloride (POCl₃=phosphoroxychloride) in the absence or presence of phosphorus pentachloride (thus giving the compound of formula IV wherein L is Cl), preferably under exclusion of moisture, if desired in the presence of (preferably lower than stoichiometric amounts of) a tertiary nitrogen base, such as triethylamine or pyridine. The reaction takes place in an inert solvent or preferably (especially where the anhydride or acid halide is liquid at least at the reaction temperature or already at room temperature) in the absence of a solvent. The preferred reaction temperatures are elevated temperatures, e.g. from 50 to about 100° C. or reflux temperature.

A compound of the formula IV can preferably be obtained by reaction of a pyrazolamide compound of the formula V, 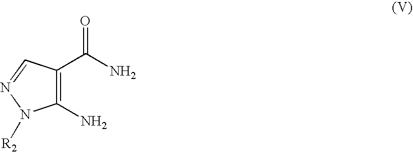
wherein R₂ is as defined for a compound of the formula I, with an amide of the formula VI,
R₃—C(═O)—NH₂ (VI)
wherein R₃ is as defined for a compound of the formula I. The reaction preferably takes place under dehydrating conditions, especially in the absence (preferred if R₃ in formula VI is hydrogen) or presence (preferred if R₃ in formula VI is substituted alkyl) of polyphosphoric acid, at preferred temperatures between 90° C. and the reflux temperature, e.g. at 100 to 195° C.

Alternatively, a compound of the formula IV wherein R₂ is as defined in formula I and R₃ is hydrogen can be prepared by reaction of a compound of the formula V wherein R₂ is as defined in formula I with tri-lower alkyl orthoformate, such as triethylorthoformate, in the presence of e.g. glacial acetic acid at elevated temperatures, e.g. between 30 and 80° C.

Still alternatively, a compound of the formula IV can directly be obtained from a compound of the formula VII given below by reaction with an acid of the formula VI*
HOOC—R₃ (VI*)
wherein R₃ is as defined for a compound of the formula I, in the presence of polyphosphoric acid at elevated temperatures, e.g. in the range from 50° C. to the reflux temperature of the reaction mixture, e.g. from 80 to 120° C.

A compound of the formula V wherein R₂ is as defined for a compound of the formula I is preferably obtained from a carbonitrile compound of the formula VII, 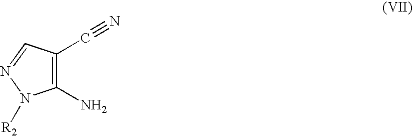
wherein R₂ is as defined for a compound of the formula I, by hydrolysis with a strong acid, preferably with concentrated (e.g. about 96%) sulfuric acid at preferred temperatures from −10° C. to about 25° C., e.g. from 0° C. to room temperature.

From a compound of the formula VII, it is also possible to directly obtain a compound of the formula IV wherein R₂ is as defined for a compound of the formula I and R₃ is hydrogen by reaction of the carbonitrile of the formula VII with formic acid at elevated temperatures, preferably under reflux conditions.

A compound of the formula VII is preferably obtained by reacting a hydrazine compound of the formula VIII,
R₂—NH—NH₂ (VIII)
wherein R₂ is as defined for a compound of the formula I, with a lower alkoxymethylenemalonitrile, preferably ethoxymethylenemalonitrile. The reaction preferably takes place in an alcohol, such as ethanol or isopropanol, in the absence or (especially where a salt form of a compound of the formula VIII is used, e.g. the hydrochloride salt) presence of a tertiary nitrogen base, e.g. a tri-lower alkylamine, such as triethylamine, at preferred temperatures from 0° C. to the reflux temperature, e.g. from room temperature to reflux temperature.

Amino compounds of the formula III, compounds of the formula VIII, as well as other starting materials are known in the art, commercially available and/or can be prepared according to standard procedures, e.g. in analogy to or by methods described in the Examples.

General Process Conditions

The following applies in general to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this disclosure only a readily removable group that is not a constituent of the particular desired end product of formula I is designated a “protecting group”, unless the context indicates otherwise. The protection of functional groups by such protecting groups, the protecting groups themselves, and the reactions appropriate for their removal are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der or ganischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps can be carried out under reaction conditions that are known Per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitrites, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in compounds of formula I described as being preferred. Special preference is given to reaction conditions that are identical or analogous to those mentioned in the Examples.

### PREFERRED EMBODIMENTS ACCORDING TO THE INVENTION

In the following preferred embodiments as well as in preceding and following embodiments of more general scope, any one or more or all general expressions can be replaced by the corresponding more specific definitions provided above and below, thus yielding stronger preferred embodiments of the invention.

A preferred embodiment of the invention relates to a compound of the formula I wherein
R₁ is a moiety of the formula Ib 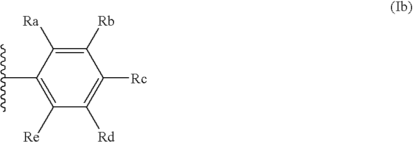
wherein Ra is methyl, ethyl, methoxy, halo or trifluoromethyl,
Re is hydrogen, methyl, ethyl, methoxy, halo or trifluoromethyl, and
Rb, Rc and Rd are independently selected from hydrogen (preferred), C₁-C₇-alkyl, C₂-C₇-alkenyl, C₂-C₇-alkynyl, hydroxy, C₁-C₇-alkoxy, amino, N-mono- or N,N-di-(C₁-C₇-alkyl)amino; halo (preferred), nitro and cyano;
R₂ is substituted phenyl wherein the substituents are one or more, preferably one or two, especially one, substituents independently selected from the group consisting of
a 3- to 8-membered heterocyclic ring, preferably bound via a ring nitrogen atom, containing, in addition to one or more carbon ring atoms, one to four nitrogen where instead of an H in —NH—C₁-C₇-alkyl may be present, oxygen or sulfur atoms (e.g. azepinyl, especially azepino, diazepinyl (such as 1,4-diazepinyl), especially diazepino, (especially N-) C₁-C₇-alkyl-diazepinyl or preferably N—C₁-C₇-alkyldiazepino, piperidinyl, especially piperidino, morpholinyl, especially morpholino, thiomorpholinyl, especially thiomorpholino, piperazinyl. especially piperazino, (especially N-) C₁-C₇-alkyl-piperazinyl, especially N—C₁-C₇-alkylpiperazino, pyrrolidinyl, especially pyrrolidino, imidazolidinyl, especially imidazolidino, (especially N-) C₁-C₇-alkyl-imidazolidinyl, preferably N—C₁-C₇-alkyl-imidazolidino, pyrazolidinyl, especially pyrazolidino, (especially N-) C₁-C₇-alkylpyrazolidinyl, preferably C₁-C₇-alkylpyrazolidino, azetidinyl, especially azetidino, or aziridinyl, especially aziridino) which ring is unsubstituted or substituted by either
(i) a 3- to 8-membered heterocyclic ring, preferably bound via a ring carbon or nitrogen atom, containing, in addition to one or more carbon ring atoms, one to four nitrogen where instead of an H in NH C₁-C₇-alkyl may be present, oxygen or sulfur atoms (e.g. azepinyl, diazepinyl (such as 1,4-diazepinyl), (especially N—) C₁-C₇-alkyl-diazepinyl, piperidinyl, morpholinyl, thiomorpholinyl, piperazinyl, (especially N-) C₁-C₇-alkyl-piperazinyl, pyrrolidinyl, imidazolidinyl, (especially N-) C₁-C₇-alkyl-imidazolidinyl, pyrazolidinyl, (especially N-) C₁-C₇-alkylpyrazolidinyl, azetidinyl or aziridinyl)
(ii) amino-C₁-C₇-alkyl or by N-mono or N,N-disubstituted amino-C₁-C₇-alkyl, wherein the amino substituents are independently selected from C₁-C₇-alkyl, C₁-C₇-alkanoyl, phenyl and phenyl-C₁-C₇-alkyl, e.g. N,N-di-(C₁-C₇-alkyl)amino-C₁-C₇-alkyl, such as N,N-dimethylamino-C₁-C₇-alkyl, or
(iii) hydroxy-C₁-C₇-alkyl, e.g. hydroxymethyl, C₁-C₇-alkoxy-C₁-C₇-alkyl, (C₁-C₇-alkoxy)-C₁-C₇-alkoxy-C₁-C₇-alkyl, C₁-C₇-alkanoyl-C₁-C₇-alkyl, phenoxy-C₁-C₇-alkyl, phenyl-C₁-C₇-alkoxy-lower alkyl, such as benzyloxy-C₁-C₇-alkyl, C₁-C₇-alkoxy-carbonyloxy-C₁-C₇-alkyl, such as tert-butoxycarbonyloxy-C₁-C₇-alkyl or phenyl-C₁-C₇-alkoxycarbonyloxy-C₁-C₇-alkyl, such as benzyloxycarbonyloxy-C₁-C₇-alkyl;
N-mono- or N,N-di-(C₁-C₇alkyl)-amino-C₁-C₇-alkyl-amino and halo;
   R₃ is hydrogen, C₁-C₇-alkyl or amino-, N-mono- or N,N-di-(C₁-C₇-alkyl)-amino-C₁-C₇-alkyl, phenyl or pyridyl; and
   R₄ is hydrogen,
   or a salt thereof.

Especially preferred is a compound of the formula I, wherein

R₁ is a moiety of the formula Ib as shown above wherein

Ra is methyl, ethyl, methoxy, halo or trifluoromethyl
Re is hydrogen, methyl, ethyl, methoxy, halo or trifluoromethyl
and Rb, Rc and Rd are independently selected from hydrogen and halo;
   R₂ is phenyl or phenyl that is substituted, especially in the 3- or 4-position, by halo, especially bromo, or preferably 4-(4-methyl-piperazin-1-yl), 4-morpholin-4-yl-, 4-(4-pyrrolidin-1-yl-piperidin-1-yl), 4-(4-morpholin-4-yl-piperidin-1-yl), 4-[4-(4-methylpiperazin-1-yl)-piperidin-1-yl, 3-[4-(4-methyl-piperazin-1-yl)-piperidin-1-yl, 4-(4-diethylamino-piperidin-1-yl), 4-(4-dipropylamino-piperidin-1-yl), 4-((R,S)-, 4-((R)- or 4-((S)-3-dimethylamino-pyrrolidin-1-yl), 4-(4-methyl-[1,4]-diazepan-1-yl), 4-[4-(1-methylpiperidin-4-yl)-piperazin-1-yl], 3-(4-methyl-piperazin-1-yl), or 2-(N,N-dimethylamino)ethylamino
   R₃ is hydrogen, C₁-C₇-alkyl, especially methyl, or amino-, N-mono- or N,N-di-(C₁-C₇-alkyl)-amino-C₁-C₇-alkyl, especially (3-dimethylamino-propyl, phenyl or pyridyl, and
   R₄ is hydrogen,
   or a (preferably pharmaceutically acceptable) salt thereof.

Very preferred is a method of treating a disease or disorder, especially a proliferative disease, that depends on inadequate activity of a protein kinase, especially a protein tyrosine kinase, more especially of one or more of those mentioned as preferred herein, comprising administering to an animal, especially a human, in need of such treatment a compound of formula I (preferably described as novel or mentioned above as for use in the diagnostic or therapeutic treatment of a warm-blooded animal), where the disease to be treated is a proliferative disease, preferably a benign or especially malignant tumor, more preferably carcinoma of the brain, kidney, liver, adrenal gland, bladder, breast, stomach (especially gastric tumors), ovaries, colon, rectum, prostate, pancreas, lung, vagina, thyroid, sarcoma, glioblastomas, multiple myeloma or gastrointestinal cancer, especially colon carcinoma or colorectal adenoma, or a tumor of the neck and head, an epidermal hyperproliferation, especially psoriasis, prostate hyperplasia, a neoplasia, especially of epithelial character, preferably mammary carcinoma, or a leukemia. Also for the treatment of atherosclerosis, thrombosis, psoriasis, scleroderma and fibrosis, the compounds of the formula I are valuable. Other diseases or disorders in the treatment of which compounds of the formula I may be of use are atherosclerotic plaque rupture, osteoarthritis, chronic respiratory diseases (e.g. COPD, asthma), glomerulonephritis, neurodegenerative diseases (e.g. Alzheimer, Parkinson) and diabetic complications.

Most preferred is a compound of the formula I, or a (preferably pharmaceutically acceptable) salt thereof, as exemplified herein-below under ‘Examples’, or its “use” as defined above.

### FURTHER EMBODIMENTS ACCORDING TO THE INVENTION

A further embodiment of the invention relates to a compound of the formula I 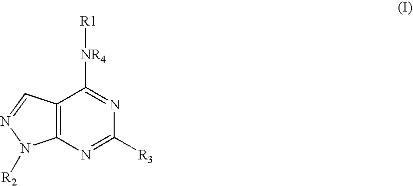
wherein R₁ is a moiety of the formula Ib 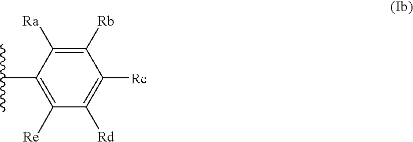
wherein Ra is methyl, ethyl, methoxy, halo or trifluoromethyl;
Re is hydrogen, methyl, ethyl, methoxy, halo or trifluoromethyl, and
Rb, Rc and Rd are independently selected from hydrogen and phenyl substituents;
R₂ is unsubstituted or substituted aryl;
R₃ is hydrogen or unsubstituted or substituted alkyl; and
R₄ is hydrogen or unsubstituted or substituted alkyl;
or a (preferably pharmaceutically acceptable) salt thereof where one or more salt-forming groups are present, for use in the diagnostic or preferably therapeutic treatment of a warm-blooded animal, especially for use in the treatment of a disease or disorder that is dependent on inadequate activity of a protein kinase, especially a protein tyrosine kinase, especially of one or more of c-Abl, c-Src and/or especially an Ephrin receptor kinase, especially EphB4 kinase; and/or one or more altered or mutated forms of any one or more of these (e.g. those that result in conversion of the respective proto-oncogene into an oncogene, such as constitutively activated Bcr-Abl or v-Src).

A further embodiment of the invention relates to a compound of the formula I wherein
R₁ is a moiety of the formula Ib 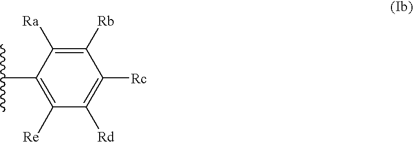
wherein Ra is methyl, ethyl, methoxy, halo or trifluoromethyl;
Re is hydrogen, methyl, ethyl, methoxy, halo or trifluoromethyl, and
Rb, Rc and Rd are independently selected from hydrogen (preferred), unsubstituted or substituted alkyl, unsubstituted or substituted alkenyl, unsubstituted or substituted alkynyl, unsubstituted or substituted aryl, unsubstituted or substituted heterocyclyl, hydroxy, esterified or etherified hydroxy, unsubstituted, mono- or disubstituted amino wherein the substituents are independently selected from unsubstituted or substituted alkyl and unsubstituted or substituted aryl; halo (preferred), nitro, cyano, mercapto, substituted mercapto, sulfo and substituted sulfonyl wherein the substituents are selected from unsubstituted or substituted alkyl and unsubstituted or substituted aryl;
R₂ is unsubstituted or preferably substituted aryl;
R₃ is hydrogen or unsubstituted or substituted alkyl; and
R₄ is hydrogen or unsubstituted or substituted alkyl,
with the proviso that if R₂ is 4-methoxyphenyl, R₃ is hydrogen and R₄ is hydrogen, then R₁ is a moiety falling under the definition of R₁ other than 5-fluoro-2-methylphenyl and 2-methylphenyl; and with the proviso that R₁ is a moiety falling under the definition of R₁ other than unsubstituted or substituted 3-nitrophenyl;

A further embodiment of the invention relates to a compound of the formula I wherein
R₁ is a moiety of the formula Ib 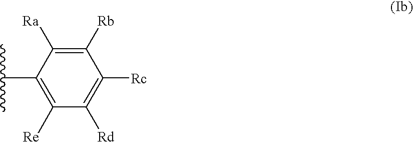
wherein Ra is methyl, ethyl, methoxy, halo or trifluoromethyl;
Re is hydrogen, methyl, ethyl, methoxy, halo or trifluoromethyl, and
Rb, Rc and Rd are independently selected from hydrogen (preferred), C₁-C₇-alkyl, C₂-C₇-alkenyl, C₂-C₇-alkynyl, hydroxy, C₁-C₇-alkoxy, amino, N-mono- or N,N-di-(C₁-C₇-alkyl)amino; halo (preferred), nitro and cyano;
R₂ is substituted phenyl wherein the substituents are one or more, preferably one or two, especially one, substituents independently selected from the group consisting of
a 3- to 8-membered heterocyclic ring, preferably bound via a ring nitrogen atom, containing, in addition to one or more carbon ring atoms, one to four nitrogen where instead of an H in —NH—C₁-C₇-alkyl may be present, oxygen or sulfur atoms (e.g. azepinyl, especially azepino, diazepinyl (such as 1,4-diazepinyl), especially diazepino, (especially N-) C₁-C₇-alkyl-diazepinyl or preferably N—C₁-C₇-alkyldiazepino, piperidinyl, especially piperidino, morpholinyl, especially morpholino, thiomorpholinyl, especially thiomorpholino, piperazinyl. especially piperazino, (especially N-) C₁-C₇-alkyl-piperazinyl, especially N—C₁-C₇-alkyl-piperazino, pyrrolidinyl, especially pyrrolidino, imidazolidinyl, especially imidazolidino, (especially N-) C₁-C₇-alkyl-imidazolidinyl, preferably N—C₁-C₇-alkyl-imidazolidino, pyrazolidinyl, especially pyrazolidino, (especially N-) C₁-C₇-alkylpyrazolidinyl, preferably C₁-C₇-alkylpyrazolidino, azetidinyl, especially azetidino, or aziridinyl, especially aziridino) which ring is unsubstituted or substituted by either
   (j) a 3- to 8-membered heterocyclic ring, preferably bound via a ring carbon or nitrogen atom, containing, in addition to one or more carbon ring atoms, one to four nitrogen where instead of an H in NH C₁-C₇-alkyl may be present, oxygen or sulfur atoms (e.g. azepinyl, diazepinyl (such as 1,4-diazepinyl), (especially N-) C₁-C₇-alkyl-diazepinyl, piperidinyl, morpholinyl, thiomorpholinyl, piperazinyl, (especially N-) C₁-C₇-alkyl-piperazinyl, pyrrolidinyl, imidazolidinyl, (especially N-) C₁-C₇-alkyl-imidazolidinyl, pyrazolidinyl, (especially N-) C₁-C₇-alkylpyrazolidinyl, azetidinyl or aziridinyl)
   (ii) amino-C₁-C₇-alkyl or by N-mono or N,N-disubstituted amino-C₁-C₇-alkyl, wherein the amino substituents are independently selected from C₁-C₇-alkyl, C₁-C₇-alkanoyl, phenyl and phenyl-C₁-C₇-alkyl, e.g. N,N-di-(C₁-C₇-alkyl)amino-C₁-C₇-alkyl, such as N,N-dimethylamino-C₁-C₇-alkyl, or
   (iii) hydroxy-C₁-C₇-alkyl, e.g. hydroxymethyl, C₁-C₇-alkoxy-C₁-C₇-alkyl, (C₁-C₇-alkoxy)-C₁-C₇-alkoxy-C₁-C₇-alkyl, C₁-C₇-alkanoyl-C₁-C₇-alkyl, phenoxy-C₁-C₇-alkyl, phenyl-C₁-C₇-alkoxy-lower alkyl, such as benzyloxy-C₁-C₇-alkyl, C₁-C₇-alkoxy-carbonyloxy-C₁-C₇-alkyl, such as tert-butoxycarbonyloxy-C₁-C₇-alkyl or phenyl-C₁-C₇-alkoxycarbonyloxy-C₁-C₇-alkyl, such as benzyloxycarbonyloxy-C₁-C₇-alkyl;
and halo;
   R₃ is hydrogen, C₁-C₇-alkyl or amino-, N-mono- or N,N-di-(C₁-C₇-alkyl)-amino-C₁-C₇-alkyl; and
   R₄ is hydrogen,
   or a (preferably pharmaceutically acceptable) salt thereof; or the use of such compound and/or salt.

A further embodiment of the invention relates to a compound of the formula I, wherein

R₁ is a moiety of the formula Ib as shown above wherein

Ra is methyl, ethyl, methoxy, halo or trifluoromethyl
Re is hydrogen, methyl, ethyl, methoxy, halo or trifluoromethyl
and Rb, Rc and Rd are independently selected from hydrogen and halo;
   R₂ is phenyl or phenyl that is substituted, especially in the 3- or 4-position, by halo, especially bromo, or preferably 4-(4-methyl-piperazin-1-yl), 4-morpholin-4-yl-, 4-(4-pyrrolidin-1-yl-piperidin-1-yl), 4-(4-morpholin-4-yl-piperidin-1-yl), 4-[4-(4-methyl-piperazin-1-yl)-piperidin-1-yl, 4-(4-diethylamino-piperidin-1-yl), 4-(4-dipropylamino-piperidin-1-yl), 4-((R,S)-, 4-((R)- or 4-((S)-3-dimethylamino-pyrrolidin-1-yl), 4-(4-methyl-[1,4]-diazepan-1-yl), 4-[4-(1-methyl-piperidin-4-yl)-piperazin-1-yl] or 3-(4-methyl-piperazin-1-yl),
   R₃ is hydrogen, C₁-C₇-alkyl, especially methyl, or amino-, N-mono- or N,N-di-(C₁-C₇-alkyl)-amino-C₁-C₇-alkyl, especially (3-dimethylamino-propyl, and
   R₄ is hydrogen,
   or a (preferably pharmaceutically acceptable) salt thereof.
   Pharmaceutical Compositions

The invention relates also to pharmaceutical compositions comprising a (preferably novel) compound of formula I, to their use in the therapeutic (in a broader aspect of the invention also prophylactic) treatment or a method of treatment of a disease or disorder that depends on inadequate protein (especially tyrosine) kinase activity, especially the preferred disorders or diseases mentioned above, to the compounds for said use and to pharmaceutical preparations and their manufacture, especially for said uses. More generally, pharmaceutical preparations are useful in case of compounds of the formula I.

The pharmacologically acceptable compounds of the present invention may be present in or employed, for example, for the preparation of pharmaceutical compositions that comprise an effective amount of a compound of the formula I, or a pharmaceutically acceptable salt thereof, as active ingredient together or in admixture with one or more inorganic or organic, solid or liquid, pharmaceutically acceptable carriers (carrier materials).

The invention relates also to a pharmaceutical composition that is suitable for administration to a warm-blooded animal, especially a human (or to cells or cell lines derived from a warm-blooded animal, especially a human, e.g. lymphocytes), for the treatment (this, in a broader aspect of the invention, also including prevention of (=prophylaxis against)) a disease that responds to inhibition of protein (especially tyrosine) kinase activity, comprising an amount of a compound of formula I or a pharmaceutically acceptable salt thereof, preferably which is effective for said inhibition, together with at least one pharmaceutically acceptable carrier.

The pharmaceutical compositions according to the invention are those for enteral, such as nasal, rectal or oral, or parenteral, such as intramuscular or intravenous, administration to warm-blooded animals (especially a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The invention relates also to method of treatment for a disease that responds to inhibition of a disease that depends on inadequate activity of a protein (especially tyrosine) kinase; which comprises administering a prophylactically or especially therapeutically effective amount of a compound of formula I, or a pharmaceutically acceptable salt thereof, especially to a warm-blooded animal, for example a human, that, on account of one of the mentioned diseases, requires such treatment.

The dose of a compound of the formula I or a pharmaceutically acceptable salt thereof to be administered to warm-blooded animals, for example humans of approximately 70 kg body weight, preferably is from approximately 3 mg to approximately 10 g, more preferably from approximately 10 mg to approximately 1.5 g, most preferably from about 100 mg to about 1000 mg/person/day, divided preferably into 1-3 single doses which may, for example, be of the same size. Usually, children receive half of the adult dose.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8-22, especially from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, p-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefossé, Paris), “Miglyol 812” (triglyceride of saturated fatty acids with a chain length of C8 to C12, Hüls AG, Germany), but especially vegetable oils, such as cottonseed oil, almond oil, olive oil, castor oil, sesame oil, soybean oil and groundnut oil.

The injection or infusion compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragée coatings or the capsule casings, for example for identification purposes or to indicate different doses of active ingredient.

A compound of the formula I may also be used to advantage in combination with other biologically active agents, preferentially with other antiproliferative agents. Such antiproliferative agents include, but are not limited to aromatase inhibitors; antiestrogens; topoisomerase I inhibitors; topoisomerase II inhibitors; microtubule active agents; alkylating agents; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; agents used in the treatment of hematologic malignancies; compounds which target, decrease or inhibit the activity of Flt-3; Hsp90 inhibitors; and temozolomide (TEMODAL®).

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON.

The term “microtubule active agent” relates to microtubule stabilizing, microtubule destabilizing agents and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, cochicine and epothilones and derivatives thereof, e.g. epothilone B or a derivative thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating agent” as used herein includes, but is not limited to, cyclophosphamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g. under the trademark HOLOXAN.

The term “histone deacetylase inhibitors” or “HDAC inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA).

The term “antineoplastic antimetabolite” includes, but is not limited to, 5-fluorouracil (5-FU); capecitabine; gemcitabine; DNA demethylating agents, such as 5-azacytidine and decitabine; methotrexate; edatrexate; and folic acid antagonists such as pemetrexed. Capecitabine can be administered, e.g., in the form as it is marketed, e.g. under the trademark XELODA. Gemcitabine can be administered, e.g., in the form as it is marketed, e.g. under the trademark GEMZAR. Also included is the monoclonal antibody trastuzumab which can be administered, e.g., in the form as it is marketed, e.g. under the trademark HERCEPTIN.

The term “platin compound” as used herein includes, but is not limited to, carboplatin, cis-platin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity and further anti-angiogenic compounds” as used herein includes, but is not limited to: protein tyrosine kinase and/or serine and/or threonine kinase inhibitors or lipid kinase inhibitors, e.g.:

a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668, and GFB-111;

b) compounds targeting, decreasing or inhibiting the activity of the fibroblast growth factor-receptors (FGFR);

c) compounds targeting, decreasing or inhibiting the activity of the insulin-like growth factor I receptor (IGF-IR), especially compounds which inhibit the IGF-IR, such as those compounds disclosed in WO 02/092599;

d) compounds targeting, decreasing or inhibiting the activity of the Trk receptor tyrosine kinase family;

e) compounds targeting, decreasing or inhibiting the activity of the Axl receptor tyrosine kinase family;

f) compounds targeting, decreasing or inhibiting the activity of the c-Met receptor;

g) compounds targeting, decreasing or inhibiting the activity of the c-Kit receptor tyrosine kinases—(part of the PDGFR family), such as compounds which target, decrease or inhibit the activity of the c-Kit receptor tyrosine kinase family, especially compounds which inhibit the c-Kit receptor, e.g. imatinib;

h) compounds targeting, decreasing or inhibiting the activity of members of the c-Abl family and their gene-fusion products (e.g. BCR-Abl kinase), such as compounds which target decrease or inhibit the activity of c-Abl family members and their gene fusion products, e.g. a N-phenyl-2-pyrimidine-amine derivative, e.g. imatinib; PD180970; AG957; NSC 680410; or PD173955 from ParkeDavis;

i) compounds targeting, decreasing or inhibiting the activity of members of the protein kinase C (PKC) and Raf family of serine/threonine kinases, members of the MEK, SRC, JAK, FAK, PDK and Ras/MAPK family members, or PI(3) kinase family, or of the PI(3)kinase-related kinase family, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; LY333531/LY379196; isochinoline compounds such as those disclosed in WO 00/09495; FTIs; PD184352 or QAN697 (a P13K inhibitor);

j) compounds targeting, decreasing or inhibiting the activity of a protein-tyrosine kinase, such as imatinib mesylate (GLIVEC/GLEEVEC) or tyrphostin. A tyrphostin is preferably a low molecular weight (Mr<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); and

k) compounds targeting, decreasing or inhibiting the activity of the epidermal growth factor family of receptor tyrosine kinases (EGFR, ErbB2, ErbB3, ErbB4 as homo- or heterodimers), such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (HerpetinR), cetuximab, Iressa, erlotinib (Tarceva™), CI-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541.

Further anti-angiogenic compounds include compounds having another mechanism for their activity, e.g. unrelated to protein or lipid kinase inhibition e.g. thalidomide (THALOMID) and TNP-470.

Compounds which target, decrease or inhibit the activity of a protein or lipid phosphatase are e.g. inhibitors of phosphatase 1, phosphatase 2A, PTEN or CDC25, e.g. okadaic acid or a derivative thereof.

Compounds which induce cell differentiation processes are e.g. retinoic acid, α- γ- or δ-tocopherol or α- γ- or δ-tocotrienol.

The term “cyclooxygenase inhibitor” as used herein includes, but is not limited to, e.g. Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA.

The term “heparanase inhibitor” as used herein refers to compounds which target, decrease or inhibit heparin sulphate degradation. The term includes, but is not limited to, PI-88.

The term “biological response modifier” as used herein refers to a lymphokine or interferons, e.g. interferon γ.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor”, e.g. L-744832, DK8G557 or R115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. PS-341 and MLN 341.

The term “matrix metalloproteinase inhibitor” or (“MMP inhibitor”) as used herein includes, but is not limited to collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “agents used in the treatment of hematologic malignancies” as used herein includes, but is not limited to FMS-like tyrosine kinase inhibitors e.g. compounds targeting, decreasing or inhibiting the activity of Flt-3; interferon, 1-b-D-arabinofuransylcytosine (ara-c) and bisulfan; and ALK inhibitors e.g. compounds which target, decrease or inhibit anaplastic lymphoma kinase.

The term “compounds which target, decrease or inhibit the activity of Flt-3” are especially compounds, proteins or antibodies which inhibit Flt-3, e.g. PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteasome pathway. Compounds targeting, decreasing or inhibiting the intrinsic ATPase activity of HSP93 are especially compounds, proteins or antibodies which inhibit the ATPase activity of HSP90 e.g., 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to trastuzumab (Herceptin™), Trastuzumab-DM1, bevacizumab (Avastin™), rituximab (Rituxan®), PRO64553 (anti-CD40) and 2C4 Antibody. By antibodies is meant e.g. intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of formula I can be used in combination with standard leukemia therapies, especially in combination with therapies used for the treatment of AML. In particular, compounds of formula I can be administered in combination with e.g. farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of the formula I, can be prepared and administered as described in the art such as in the documents cited above.

A compound of the formula I may also be used to advantage in combination with known therapeutic processes, e.g., the administration of hormones or especially radiation.

A compound of formula I may in particular be used as a radiosensitizer, especially for the treatment of tumors which exhibit poor sensitivity to radiotherapy.

By “combination”, there is meant either a fixed combination in one dosage unit form, or a kit of parts for the combined administration where a compound of the formula I and a combination partner may be administered independently at the same time or separately within time intervals that especially allow that the combination partners show a cooperative, e.g. synergistic, effect, or any combination thereof.

### EXAMPLES

The following Examples serve to illustrate the invention without limiting its scope. Temperatures are measured in degrees Celsius. Unless otherwise indicated, the reactions take place at RT.

The R_{f} values in TLC indicate the ratio of the distance moved by each substance to the distance moved by the eluent front. R_{f} values for TLC are measured on 5×10 cm TLC plates, silica gel F₂₅₄, Meck, Darmstadt, Germany; the solvent system used is 20% hexane/80% (tert-butylmethylether with 2% triethylamine). Further solvent systems for R_{f} values marked are:

* 5% Hexanes/95% (tert-butylmethylether with 5% triethylamine)

** 75% (Ethylacetate with 5% triethylamine)/25% methanol

*** 20% Hexanes/80% (ethylacetate with 5% triethylamine)

If not indicated otherwise, the analytical HPLC conditions are as follows:
Column: (70×3 mm) packed with reversed-phase material Nucleosil 100-3 C18 (Macherey & Nagel, Düren, Germany). Detection by UV absorption at 220 and 254 nm. The retention times (t_{R}) are given in minutes. Flow rate: 1.8 mL/min.
Gradient: 5%→100% A in B for 4 min+0.4 min 100% A. A: Acetonitrile; B: water+0.1% TFA.

Method Indicated with ¹:
Column: (50×4.6 mm) packed with reversed-phase material 5 μm XTerra 100-3 C18 (Waters Corp., Milford, Mass., U.S.A.). Detection by UV absorption at 220 and 254 nm. The retention times (t_{R}) are given in minutes. Flow rate: 2 mL/min.
Gradient: 5%→100% A in B for 4 min+0.4 min 100% A. A: Acetonitrile+0.07% formic acid; B: water+0.1% formic acid.

Method indicated with ²:
Column: (50×4.6 mm) packed with reversed-phase material 5 μm XTerra 100-3 C18 (Waters Corp., Milford, Mass., U.S.A.). Detection by UV absorption at 220 and 254 nm. The retention times (t_{R}) are given in minutes. Flow rate: 2 mL/min.
Gradient: 5%→100% A in B for 8 min+1.5 min 100% A. A: Acetonitrile+0.07% formic acid; B: water+0.1% formic acid.

Method indicated with ³:
Column: Column Engineering, Inc., Matrix, 3 μm C18 150×4.6 mm (Lot# 205) Detection by UV absorption at 215 and 254 nm. The column temperature is 35° C. and the retention times (tR) are given in minutes. Flow rate: 1 mL/min.
Gradient: water (0.1% TFA)/acetonitrile (0.1% TFA)=98/2 for 1 min. To 100% acetonitrile (0.1% TFA) in 10 min. Stay at 100% for 2 min (total run time: 13 min.)

The short forms and abbreviations used have the following meanings:

ACN acetonitrile

tBuOH tert.-Butanol

conc. concentrated

DMF N,N-dimethylformamide

DMA N,N-dimethylacetamide

EtOH Ethanol

eq. equivalent(s)

HPLC High Performance Liquid Chromatography

MPLC Medium Pressure Liquid Chromatography

MS-ES mass spectroscopy (electron spray)

h hour(s)

Me methyl

min minute(s)

iPrOH Isopropanol

RP Reversed Phase

RT room temperature

TEA triethylamine

TFA trifluoroacetic acid (in salt: trifluoroacetate)

THF tetrahydrofuran (distilled over Na/benzophenone)

TLC thin-layer chromatography

t_{R} retention times

### Example 1

### {1-[4-(4-Methyl-piperazin-1-yl)-phenyl]-1H-pyrazolo[3,4-d]pyrimidin-4-yl}-o-tolyl-amine•3 TFA

[1-(4-Bromo-phenyl)-1H-pyrazolo[3,4-d]pyrimidin-4-yl]-o-tolyl-amine (50 mg, 0.12 mmol), N-methylpiperazine (30 μL, 0.24 mmol), and potassium tert-butoxide (60 mg, 0.48 mmol) are added to 1 mL of degassed THF under an atmosphere of argon. As palladium catalyst, 2-(dimethylamino)ferrocen-1-yl-palladium(II) chloride dinorbornyl-phosphine complex (8 mg; 0.012 mmol) is added and the reaction mixture is stirred at 110° C. for 20 min. The THF is evaporated and the remaining residue is dissolved in DMA. The obtained suspension is filtered and the solution is purified by MPLC:
Column: 70 g of reversed-phase material POLYGOPREP 60-50 C18 (GFS Chemicals, Inc., Powell, Ohio). Detection by UV absorption at 254 nm. Flow rate: 30 mL/min.
Gradient: 0 to 2 min: 20% B. 2 to 15 min: 20 to 40% B. 15 to 17 min: 40% B. 17 to 18 min: 40 to 95% B. 18 to 19 min: 95% B. A: Water+0.1% TFA; B: Acetonitrile+0.1% TFA.

Pure fractions are pooled, the ACN is removed under reduced pressure, the water is removed by freeze-drying, the remainder is dissolved in tBuOH and freeze-dried to give the title compound as a brown solid. HPLC t_{R}=1.80 min; MS-ES+: (M+H)+=400. ¹H-NMR (400 MHz, CDCl₃): δ (ppm)=13.50 (s, 1H), 8.33 (s, 1H), 7.85 (d, 2H), 7.35-7.45 (m, 4H), 7.03 (d, 2H), 6.80 (s, 2H), 3.70 (s, 4H), 3.45 (s, 4H), 2.90 (s, 3H), 2.35 (s, 3H). R_{f}*=0.11.

The starting materials are prepared as follows:

### Step 1.1: [1-(4-Bromo-Phenyl)-1H-pyrazolo[3,4-d]pyrimidin-4-yl]-o-tolyl-amine•TFA

1-(4-Bromo-phenyl)-4-chloro-1H-pyrazolo[3,4-d]pyrimidine (300 mg, 0.97 mmol) is dissolved in 2 mL of 3-pentanol. o-Toluidine (110 μL, 1.02 mmol) is added and the mixture is stirred at 150° C. for 15 min in a microwave reactor. The 3-pentanol is evaporated, the residue is dissolved in DMA and purified by RP-HPLC:
Column: 5 μm, 19×50 mm, packed with reverse-phase material X-Terra RP18 (Waters, Milford, Mass.)
   Detection by UV absorption at 220 nm. Flow rate: 20 mL/min.
Gradient: 0 to 1.5 min: 30% B. 1.5 to 4 min: 30 to 65% B. 4 to 7 min: 65% B. 7 to 9 min: 65 to 95% B. 9 to 10 min: 95% B. (A and B as in Example 1).

Pure fractions are pooled, the ACN is removed under reduced pressure, the water is removed by freeze-drying to give the title compound as an off-white powder.

HPLC t_{R}=2.93 min; MS-ES+: (M+H)+=381.

### Step 1.2: 1-(4-Bromo-phenyl)-4-chloro-1H-pyrazolo[3,4-d]pyrimidine

1-(4-Bromo-phenyl)-1,5-dihydro-pyrazolo[3,4-d]pyrimidin-4-one (3 g; 0.01 mol) is dissolved in 18 mL of phosphoroxychloride. The reaction mixture is stirred and refluxed overnight. Excess phosphoroxychloride is evaporated under reduced pressure and the obtained syrup is poured onto crushed ice. The aqueous solution is extracted with chloroform and dried over sodium sulfate. The chloroform is evaporated and the title compound is obtained as a gray solid. HPLC t_{R}=3.20 min; MS-ES+: (M+H)+=310.

### Step 1.3: 1-(4-Bromo-phenyl)-1,5-dihydro-pyrazolo[3,4-d]pyrimidin-4-one

5-Amino-1-(4-bromo-phenyl)-1H-pyrazole-4-carboxylic acid amide (5 g, 0.018 mol) is heated with 14 mL of formamide at 180° C. for 4 h. The reaction mixture is cooled at RT and set aside in a refrigerator overnight. The product is filtered, washed with water and dried. The title compound is obtained as an off-white solid. HPLC t_{R}=2.24 min; MS-ES+: (M+H)+=292.

### Step 1.4: 5-Amino-1-(4-bromo-phenyl)-1H-pyrazole-4-carboxylic acid amide

The same procedure as described in example 2 step 2.2 is used, except that 5-amino-1-(4-bromo-phenyl)-1H-pyrazole-4-carbonitrile is used. The title compound is obtained as an off-white solid. HPLC t_{R}=1.80 min; MS-ES+: (M+H)+=282.

### Step 1.5: 5-Amino-1-(4-bromo-phenyl)-1H-pyrazole-4-carbonitrile

The same procedure as described in example 2 step 2.3 is used, except that 4-bromophenylhydrazine hydrochloric acid is used together with 1 eq. TEA in EtOH. The title compound is obtained as an off-white solid. HPLC t_{R}=2.07 min; MS-ES+: (M+H)+=264.

### Example 2

### [6-(3-Dimethylamino-propyl)-1-phenyl-1H-pyrazolo[3,4-d]pyrimidin-4-yl]-o-tolyl-amine•2 TFA

6-(3-Dimethylamino-propyl)-1-phenyl-1,5-dihydro-pyrazolo[3,4-d]pyrimidin-4-one (40 mg, 0.13 mmol) is heated in 1 mL phosphoroxychloride for 1 h. The resulting solution is evaporated and coevaporated twice with toluene. 2-Toluidin (16 μL, 0.15 mmol) in 500 μL 3-pentanol is added and the mixture is heated to 100° C. for 2 h. The resulting solution is evaporated, dissolved in DMA and purified by preparative RP-HPLC. Pure fractions are pooled, the ACN is removed under reduced pressure, the water is removed by freeze-drying, the remainder is dissolved in tBuOH and freeze-dried to give the title compound as a white powder. HPLC t_{R}=2.23 min; MS-ES+: (M+H)+=387. ¹H-NMR (400 MHz, CDCl₃): δ (ppm)=11.10 (s, 1H), 7.92 (d, 2H), 7.53 (m, 2H), 7.40 (m, 2H), 7.15 (m, 3H), 6.78 (s, 1H), 2.85 (s, 6H), 2.32 (s, 3H), 1.65 (m, 2H), 0.95 (m, 4H). R_{f}*=0.32.

The starting materials are prepared as follows:

### Step 2.1: 6-(3-Dimethylamino-propyl)-1-phenyl-1,5-dihydro-pyrazolo[3,4-d]pyrimidin-4-one

5-Amino-1-phenyl-1H-pyrazole-4-carboxylic acid amide (404 mg, 2 mmol) and 4-(dimethylamino)butyric acid hydrochloride salt (337 mg, 2 mmol) are stirred in polyphosphoric acid for 8 h at 100° C. The resulting syrup is added to 50 mL water and stirred for 1 h. 30 mL 30% aqueous ammonia is added with cooling and the resulting off-white solid is filtered, washed with few mL of water and dried. HPLC t_{R}=1.54 min; MS-ES+: (M+H)+=298.4.

### Step 2.2: 5-Amino-1-phenyl-1H-pyrazole-4-carboxylic acid amide

5-Amino-1-phenyl-1H-pyrazole-4-carbonitrile (10 g), 54 mmol) are added in portions to conc. sulfuric acid at 0° C. and stirred 1 h at RT. The resulting solution is poured on 200 g crushed ice and 90 mL 30% aqueous ammonia is added. The resulting off-white solid is filtered, washed with few mL of water and dried. HPLC t_{R}=1.30 min; MS-ES+: (M+H)+=203.3.

### Step 2.3: 5-Amino-1-phenyl-1H-pyrazole-4-carbonitrile

A suspension of 10.81 g phenylhydrazin (0.1 mol) and 12.25 g ethoxymethylenemalononitril in 50 mL iPrOH are stirred for 1 h at RT. The resulting solution is cooled to 4° C. over night, the resulting off-white crystals are filtered, washed with few mL of ice cold iPrOH and dried.

HPLC t_{R}=1.61 min; MS-ES+: (M+H)+=185.3.

### Example 3

### [1-(4-Morpholin-4-yl-phenyl)-1H-pyrazolo[3,4-d]pyrimidin-4-yl]-o-tolylamine•2 TFA

The same procedure as described in example 1 is used, except that morpholine is used instead of N-methylpiperazine. The title compound is obtained as an off-white solid.

HPLC t_{R}=2.26 min; MS-ES+: (M+H)+=387. R_{f}=0.35.

### Example 4

### (2,6-Dimethyl-Phenyl)-{1-[4-(4-methyl-piperazin-1-yl)-phenyl]-1H-pyrazolo[3,4-d]pyrimidin-4-yl}-amine•3 TFA

The same procedure as described in example 1 is used, except that in step 1.1 2,6-dimethylaniline is used. The title compound is obtained as an off-white solid. HPLC t_{R}=1.88 min; MS-ES+: (M+H)+=414. R_{f}*=0.12.

### Example 5

### {1-[3-(4-Methyl-piperazin-1-yl)-phenyl]-1H-pyrazolo[3,4-d]pyrimidin-4-yl}-o-tolyl-amine•3 TFA

The same procedure as described in example 1 is used, except that in step 1.5 3-bromophenylhydrazine hydrochloric acid is used together with 1 eq. TEA in EtOH. The title compound is obtained as an off-white solid. HPLC t_{R}=1.87 min; MS-ES+: (M+H)+=400. R_{f}*=0.16.

### Example 6

### (2,6-Dimethyl-Phenyl)-{1-[3-(4-methyl-piperazin-1-yl)-phenyl]-1H-pyrazolo[3,4-d]pyrimidin-4-yl}-amine•3 TFA

The same procedure as described in example 1 is used, except that in step 1.5 3-bromophenylhydrazine hydrochloric acid is used together with 1 eq. TEA in EtOH and in step 1.1 2,6-dimethylaniline is used. The title compound is obtained as an off-white solid. HPLC t_{R}=1.97 min; MS-ES+: (M+H)+=414. R_{f}*=0.16.

### Example 7

### {1-[4-(4-Pyrrolidin-1-yl-piperidin-1-yl)-phenyl]-1H-pyrazolo[3,4-d]pyrimidin-4-yl}-o-tolyl-amine•3 TFA

The same procedure as described in example 1 is used, except that 4-pyrrolidin-1-yl-piperidine is used instead of N-methylpiperazine. The title compound is obtained as an off-white solid. HPLC t_{R}=1.95 min; MS-ES+: (M+H)+=454. R_{f}=0.13.

### Example 8

### (2,6-Dimethyl-phenyl)-{1-[4-(4-pyrrolidin-1-yl-piperidin-1-yl)-phenyl]-1H-pyrazolo[3,4-d]pyrimidin-4-yl}-amine•3 TFA

The same procedure as described in example 1 is used, except that 4-pyrrolidin-1-yl-piperidine is used instead of N-methylpiperazine and in step 1.1 2,6-dimethylaniline is used instead of o-toluidine. The title compound is obtained as an off-white solid. HPLC t_{R}=2.02 min; MS-ES+: (M+H)+=468. R_{f}*=0.13.

### Example 9

### {1-[4-(4-Morpholin-4-yl-piperidin-1-yl)-phenyl]-1H-pyrazolo[3,4-d]pyrimidin-4-yl}-o-tolyl-amine•3 TFA

The same procedure as described in example 1 is used, except that 4-piperidin-4-yl-morpholine is used instead of N-methylpiperazine. The title compound is obtained as an off-white solid. HPLC t_{R}=1.84 min; MS-ES+: (M+H)+=470. R_{f}*=0.16.

### Example 10

### (2,6-Dimethyl-Phenyl)-{1-[4-(4-morpholin-4-yl-piperidin-1-yl)-phenyl]-1H-pyrazolo[3,4-d]pyrimidin-4-yl}-amine•3 TFA

The same procedure as described in example 1 is used, except that 4-piperidin-4-yl-morpholine is used instead of N-methylpiperazine and in step 1.1 2,6-dimethylaniline is used instead of o-toluidine. The title compound is obtained as an off-white solid. HPLC t_{R}=1.94 min; MS-ES+: (M+H)+=484. R_{f}*=0.16.

### Example 11

### (1-{4-[4-(4-Methyl-piperazin-1-yl)-piperidin-1-yl]-phenyl}-1H-pyrazolo[3,4-d]pyrimidin-4-yl)-o-tolyl-amine•4 TFA

The same procedure as described in example 1 is used, except that 1-methyl-4-piperidin-4-yl-piperazine is used instead of N-methylpiperazine. The title compound is obtained as an off-white solid. HPLC t_{R}=1.75 min; MS-ES+: (M+H)+=483. ¹H-NMR (400 MHz, D₆-DMSO): δ (ppm)=9.80 (s, 1H), 8.30 (s, 1H), 7.83 (d, 2H), 7.39-7.43 (m, 4H), 3.60 (m, 2H), 3.55 (s, 4H), 3.25 (s, 4H), 2.82 (s, 3H), 2.78 (m, 2H), 2.22 (s, 3H), 2.07 (m, 2H), 1.69 (m, 2H).

### Example 12

### (2,6-Dimethyl-phenyl)-(1-{4-[4-(4-methyl-piperazin-1-yl)-piperidin-1-yl]-phenyl}-1H-pyrazolo[3,4-d]pyrimidin-4-yl)-amine•4 TFA

The same procedure as described in example 1 is used, except that 1-methyl-4-piperidin-4-yl-piperazine is used instead of N-methylpiperazine and in step 1.1 2,6-dimethylaniline is used instead of o-toluidine. The title compound is obtained as an off-white solid. HPLC t_{R}=1.84 min; MS-ES+: (M+H)+=497. R_{f}*=0.32.

### Example 13

### {1-[4-(4-Diethylamino-Piperidin-1-yl)-phenyl]-1H-pyrazolo[3,4-d]pyrimidin-4-yl}-o-tolyl-amine•3 TFA

The same procedure as described in example 1 is used, except that 4-diethylaminopiperidine is used instead of N-methylpiperazine. The title compound is obtained as an off-white solid. HPLC t_{R}=1.98 min; MS-ES+: (M+H)+=456. ¹H-NMR (400 MHz, CDCl₃): δ (ppm)=13.0 (s, 1H), 8.3 (s, 1H), 7.75 (d, 2H), 7.45-7.48 (m, 4H), 7.0 (d, 2H), 6.65 (s, 1H), 3.85 (m, 4H), 3.10 (m, 1H), 2.90 (m, 4H), 2.45 (s, 3H), 2.10 (m, 10H). R_{f}*=0.14.

### Example 14

### {1-[4-(4-Di-n-propylamino-piperidin-1-yl)-phenyl]-1H-pyrazolo[3,4-d]pyrimidin-4-yl}-o-tolyl-amine•3 TFA

The same procedure as described in example 1 is used, except that 4-dipropylamino-piperidine is used instead of N-methylpiperazine. The title compound is obtained as an off-white solid. HPLC t_{R}=2.18 min; MS-ES+: (M+H)+=484. R_{f}=0.14.

### Example 15

### {1-[4-((R)-3-Dimethylamino-pyrrolidin-1-yl)-phenyl]-1H-pyrazolo[3,4-d]pyrimidin-4-y}-o-tolyl-amine•3 TFA

The same procedure as described in example 1 is used, except that (R)-3-dimethylamino-pyrrolidin is used instead of N-methylpiperazine. The title compound is obtained as an off-white solid. HPLC t_{R}=1.85 min; MS-ES+: (M+H)+=414. R_{f}=0.67.

### Example 16

### {1-[4-((S)-3-Dimethylamino-pyrrolidin-1-yl)-phenyl]-1H-pyrazolo[3,4-d]pyrimidin-4-yl}-o-tolyl-amine•3 TFA

The same procedure as described in example 1 is used, except that (S)-3-dimethylamino-pyrrolidin is used instead of N-methylpiperazine. The title compound is obtained as an off-white solid. HPLC t_{R}=1.90 min; MS-ES+: (M+H)+=414. R_{f}=0.67.

### Example 17

### {1-[4-(4-Methyl-[1,4]-diazepan-1-yl)-phenyl]-1H-pyrazolo[3,4-d]pyrimidin-4-yl}-o-tolyl-amine•3 TFA

The same procedure as described in example 1 is used, except that 4-methyl-[1,4]diazepane is used instead of N-methylpiperazine. The title compound is obtained as an off-white solid. HPLC t_{R}=1.84 min; MS-ES+: (M+H)+=414. R_{f}*=0.12

### Example 18

### (1-{4-[4-(1-Methyl-piperidin-4-yl)-piperazin-1-yl]-phenyl}-1H-pyrazolo[3,4-d]pyrimidin-4-yl)-o-tolyl-amine•4 TFA

The same procedure as described in example 1 is used, but 4-(1-methyl-piperidin-4-yl)-piperazine is used instead of N-methylpiperazine. The title compound is obtained as an off-white solid. HPLC t_{R}=1.64 min; MS-ES+: (M+H)+=483. R_{f}=0.32.

### Example 19

### {6-Methyl-1-[4-(4-methyl-piperazin-1-yl)-phenyl]-1H-pyrazolo[3,4-d]pyrimidin-4-yl}-o-tolyl-amine•3 TFA

The same procedure as described in example 1 is used, except that instead of step 1.3 step 2.1 (example 2) with acetic acid instead of 4-(dimethylamino)butyric acid hydrochloride salt is used. The title compound is obtained as an off-white solid. HPLC t_{R}=1.83 min; MS-ES+: (M+H)+=414. R_{f}*=0.11

### Example 20

### {6-Methyl-1-[3-(4-methyl-piperazin-1-yl)-phenyl]-1H-pyrazolo[3,4-d]pyrimidin-4-yl}-o-tolyl-amine•3 TFA

The same procedure as described in example 5 is used, except that instead of step 1.3 step 2.1 (example 2) with acetic acid instead of 4-(dimethylamino)butyric acid hydrochloride salt is used. The title compound is obtained as an off-white solid. HPLC t_{R}=1.84 min; MS-ES+: (M+H)+=414. R_{f}*=0.11

### Example 21

### [1-(4-Methoxy-phenyl)-1H-pyrazolo[3,4-d]pyrimidin-4-yl]-o-tolyl-amine•TFA

The same procedure as described in example 1 steps 1.5 to 1.1 is used, except that in step 1.5 4-methoxyphenylhydrazine hydrochloric acid is used. The title compound is obtained as a white solid. HPLC t_{R}=2.37 min; MS-ES+: (M+H)+=332.

### Example 22

### (2,6-Dimethyl-phenyl)-[1-(4-methoxy-phenyl)-1H-pyrazolo[3,4-d]pyrimidin-4-yl]-amine•TFA

The same procedure as described in example 1 steps 1.5 to 1.1 is used, except that in step 1.5 4-methoxyphenylhydrazine hydrochloric acid is used and in step 1.1 2,6-dimethylaniline is used instead of o-toluidine. The title compound is obtained as a white solid. HPLC t_{R}=2.48 min; MS-ES+: (M+H)+=346. R_{f}=0.63.

### Example 23

### (5-Fluoro-2-methyl-phenyl)-[1-(4-methoxy-phenyl)-1H-pyrazolo[3,4-d]pyrimidin-4-yl]-amine•TFA

The same procedure as described in example 1 steps 1.5 to 1.1 is used, except that in step 1.5 4-methoxyphenylhydrazine hydrochloric acid is used and in step 1.1 5-fluoro-2-methylaniline is used instead of o-toluidine. The title compound is obtained as a white solid. HPLC t_{R}=2.53 min; MS-ES+: (M+H)+=350. R_{f}=0.64

### Example 24

### [1-(3-Methoxy-phenyl)-1H-pyrazolo[3,4-d]pyrimidin-4-yl]-o-tolyl-amine•TFA

The same procedure as described in example 1 steps 1.5 to 1.1 is used, except that in step 1.5 3-methoxyphenylhydrazine hydrochloric acid is used. The title compound is obtained as a white solid. HPLC t_{R}=2.40 min; MS-ES+: (M+H)+=332. R_{f}=0.67.

### Example 25

### (2,6-Dimethyl-phenyl)-[1-(3-methoxy-phenyl)-1H-pyrazolo[3,4-d]pyrimidin-4-yl]-amine•TFA

The same procedure as described in example 1 steps 1.5 to 1.1 is used, except that in step 1.5 3-methoxyphenylhydrazine hydrochloric acid is used and in step 1.1 2,6-dimethylaniline is used instead of o-toluidine. The title compound is obtained as a white solid. HPLC t_{R}=2.43 min; MS-ES+: (M+H)+=446. R_{f}=0.67.

### Example 26

### (6-Methyl-1-phenyl-1H-pyrazolo[3,4-d]pyrimidin-4-yl)-o-tolyl-amine•TFA

The same procedure as described in example 2 is used except that in step 2.1 acetic acid is used instead of 4-(dimethylamino)butyric acid hydrochloride salt. The title compound is obtained as a white solid. HPLC t_{R}=2.46 min; MS-ES+: (M+H)+=316. R_{f}=0.78.

### Example 27

### [6-(3-Dimethylamino-propyl)-1-phenyl-1H-pyrazolo[3,4-d]pyrimidin-4-yl]-(2,6-dimethyl-phenyl)-amine•2 TFA

The same procedure as described in example 2 is used except that 2,6-dimethylaniline is used instead of o-toluidine. The title compound is obtained as a white solid. HPLC t_{R}=2.30 min; MS-ES+: (M+H)+=401. R_{f}=0.75

### Example 28

### [6-(3-Dimethylamino-propyl)-1-phenyl-1H-pyrazolo[3,4-d]pyrimidin-4-yl]-(2-chlor-phenyl)-amine•2 TFA

The same procedure as described in example 2 is used except that 2-chloraniline is used instead of o-toluidine. The title compound is obtained as a white solid. HPLC t_{R}=2.32 min; MS-ES+: (M+H)+=407. R_{f}=0.75.

### Example 29

### {1-[4-(4-diethylamino-piperidin-1-yl)-phenyl]-1H-pyrazolo[3,4-d]pyrimidin-4-yl}-(2,6-dimethyl-phenyl)-amine•3 TFA

The same procedure as described in example 1 is used, except that 4-(diethylamino)-piperidine is used instead of N-methylpiperazine and in step 1.1 2,6-dimethylaniline is used instead of o-toluidine. The title compound is obtained as an off-white solid. HPLC t_{R}¹=2.91 min; MS-ES+: (M+H)+=470. R_{f}***=0.25.

### Example 30

### (2,6-Dimethyl-phenyl)-{1-[4-(4-dipropylamino-piperidin-1-yl)-phenyl]-1H-Pyrazolo[3,4-d]pyrimidin-4-yl}-amine•3 TFA

The same procedure as described in example 1 is used, except that 4-(diproylamino)-piperidine is used instead of N-methylpiperazine and in step 1.1 2,6-dimethylaniline is used instead of o-toluidine. The title compound is obtained as an off-white solid. HPLC t_{R}¹=3.21 min; MS-ES+: (M+H)+=498. R_{f}***=0.33.

### Example 31

### (2,6-Dimethyl-phenyl)-{1-[4-((R)-3-dimethylamino-pyrrolidin-1-yl)phenyl]-1H-pyrazolo[3,4-d]pyrimidin-4-yl}-amine•3 TFA

The same procedure as described in example 1 is used, except that (R)-3-dimethylamino-pyrrolidin is used instead of N-methylpiperazine and in step 1.1 2,6-dimethylaniline is used instead of o-toluidine. The title compound is obtained as an off-white solid. HPLC t_{R}¹=2.46 min; MS-ES+: (M+H)+=428. R_{f}***=0.85.

### Example 32

### (2,6-Dimethyl-phenyl)-(1-{4-[4-(1-methyl-piperidin-4-yl)-piperazin-1-yl]-phenyl}-1H-pyrazolo[3,4-d]pyrimidin-4-yl)-amine•4 TFA

The same procedure as described in example 1 is used, except that 4-(1-methylpiperidin-4-yl)-piperazin-1-yl is used instead of N-methylpiperazine and in step 1.1 2,6-dimethylaniline is used instead of o-toluidine. The title compound is obtained as an off-white solid. HPLC t_{R}¹=2.21 min; MS-ES+: (M+H)+=497.

### Example 33

### (5-Fluoro-2-methyl-Phenyl)-{1-[4-(4-pyrrolidin-1-yl-piperidin-1-yl)phenyl]-1H-pyrazolo[3,4-d]pyrimidin-4-yl}-amine•3 TFA

The same procedure as described in example 1 is used, except that 4-pyrrolidin-1-yl-piperidine is used instead of N-methylpiperazine and in step 1.1 5-fluoro-2-methylaniline is used instead of o-toluidine. The title compound is obtained as an off-white solid. HPLC t_{R}¹=2.66 min; MS-ES+: (M+H)+=472. R_{f}**=0.42.

### Example 34

### (5-Fluoro-2-methyl-phenyl)-{1-[4-(4-methyl-piperazin-1-yl)-phenyl]-1H-pyrazolo[3,4-d]pyrimidin-4-yl}-amine•3 TFA

The same procedure as described in example 1 is used, except that in step 1.1 5-fluoro-2-methylaniline is used instead of o-toluidine. The title compound is obtained as an off-white solid. HPLC t_{R}¹=2.34 min; MS-ES+: (M+H)+=418. R_{f}**=0.44.

### Example 35

### (5-Fluoro-2-methyl-phenyl)-(1-{4-[4-(4-methyl-piperazin-1-yl)-piperidin-1-yl]-phenyl}-1H-pyrazolo-[3,4-d]pyrimidin-4-yl)-amine•4 TFA

The same procedure as described in example 1 is used, except that 4-(4-methylpiperazin-1-yl)-piperidine is used instead of N-methylpiperazine and in step 1.1 5-fluoro-2-methylaniline is used instead of o-toluidine. The title compound is obtained as an off-white solid. HPLC t_{R}¹=2.32 min; MS-ES+: (M+H)+=501. R_{f}**=0.31.

### Example 36

### {1-[4-(4-Diethylamino-piperidin-1-yl)-phenyl]-1H-pyrazolo[3,4-d]pyrimidin-4-yl}-(5-fluoro-2-methyl-phenyl)-amine•3 TFA

The same procedure as described in example 1 is used, except that 4-diethylaminopiperidine is used instead of N-methylpiperazine and in step 1.1 5-fluoro-2-methylaniline is used instead of o-toluidine. The title compound is obtained as an off-white solid. HPLC t_{R}¹=2.71 min; MS-ES+: (M+H)+=474. R_{f}**=0.39.

### Example 37

### {1-[4-(4-Diproylamino-piperidin-1-yl)-phenyl]-1H-pyrazolo[3,4-d]pyrimidin-4-yl}-(5-fluoro-2-methyl-phenyl)-amine•3 TFA

The same procedure as described in example 1 is used, except that 4-diproylamino-piperidine is used instead of N-methylpiperazine and in step 1.1 5-fluoro-2-methylaniline is used instead of o-toluidine. The title compound is obtained as an off-white solid. HPLC t_{R}¹=2.78 min; MS-ES+: (M+H)+=502. R_{f}**=0.58.

### Example 38

### {1-[4-((R)-3-Dimethylamino-pyrrolidin-1-yl)-phenyl]-1H-pyrazolo[3,4-d]pyrimidin-4-yl}-(5-fluoro-2-methyl-phenyl)-amine•3 TFA

The same procedure as described in example 1 is used, except that (R)-3-dimethylamino-pyrrolidin-1-yl is used instead of N-methylpiperazine and in step 1.1 5-fluoro-2-methylaniline is used instead of o-toluidine. The title compound is obtained as an off-white solid. HPLC t_{R}¹=2.46 min; MS-ES+: (M+H)+=432. R_{f}**=0.40.

### Example 39

### {1-[4-((S)-3-Dimethylamino-pyrrolidin-1-yl)-phenyl]-1H-pyrazolo[3,4-d]pyrimidin-4-yl}-(5-fluoro-2-methyl-phenyl)-amine•3 TFA

The same procedure as described in example 1 is used, except that (S)-3-dimethylamino-pyrrolidin-1-yl is used instead of N-methylpiperazine and in step 1.1 5-fluoro-2-methylaniline is used instead of o-toluidine. The title compound is obtained as an off-white solid. HPLC t_{R}¹=2.53 min; MS-ES+: (M+H)+=432.

### Example 40

### (5-Fluoro-2-methyl-phenyl)-(1-{4-[4-(1-methyl-piperidin-4-yl)-piperazin-1-yl]-phenyl}-1H-pyrazolo[3,4-d]pyrimidin-4-yl)-amine•4 TFA

The same procedure as described in example 1 is used, except that 4-(1-methylpiperidin-4-yl)-piperazine is used instead of N-methylpiperazine and in step 1.1 5-fluoro-2-methylaniline is used instead of o-toluidine. The title compound is obtained as an off-white solid. HPLC t_{R}¹=1.90 min; MS-ES+: (M+H)+=501. R_{f}**=0.19.

### Example 41

### (2-Chloro-phenyl)-{1-[4-(4-pyrrolidin-1-yl-piperidin-1-yl)-phenyl]-1H-pyrazolo[3,4-d]pyrimidin-4-yl}-amine•3 TFA

The same procedure as described in example 1 is used, except that 4-pyrrolidin-1-yl-piperidine is used instead of N-methylpiperazine and in step 1.1 2-chloraniline is used instead of o-toluidine. The title compound is obtained as an off-white solid. HPLC t_{R}¹=2.66 min; MS-ES+: (M+H)+=474. R_{f}**=0.38.

### Example 42

### (2-Chloro-phenyl)-{1-[4-(4-methyl-piperazin-1-yl)-phenyl]-1H-pyrazolo[3,4-d]pyrimidin-4-yl}-amine•3 TFA

The same procedure as described in example 1 is used, except that in step 1.1 2-chloraniline is used instead of o-toluidine. The title compound is obtained as an off-white solid. HPLC t_{R}²=3.28 min; MS-ES+: (M+H)+=420. R_{f}**=0.41.

### Example 43

### (2-Chloro-phenyl)-(1-{4-[4-(4-methyl-piperazin-1-yl)-piperidin-1-yl]-phenyl}-1H-pyrazolo[3,4-d]pyrimidin-4-yl)-amine•4 TFA

The same procedure as described in example 1 is used, except that 4-(4-methylpiperazin-1-yl)-piperidine is used instead of N-methylpiperazine and in step 1.1 2-chloraniline is used instead of o-toluidine. The title compound is obtained as an off-white solid. HPLC t_{R}¹=2.17 min; MS-ES+: (M+H)+=503.

### Example 44

### (2-Chloro-phenyl)-{1-[4-(4-diethylamino-piperidin-1-yl)-phenyl]-1H-pyrazolo[3,4-d]pyrimidin-4-yl}-amine•3 TFA

The same procedure as described in example 1 is used, except that 4-diethylaminopiperidine is used instead of N-methylpiperazine and in step 1.1 2-chloraniline is used instead of o-toluidine. The title compound is obtained as an off-white solid. HPLC t_{R}¹=2.72 min; MS-ES+: (M+H)+=476.

### Example 45

### (2-Chloro-phenyl)-{1-[4-(4-diproylamino-piperidin-1-yl)-phenyl]-1H-pyrazolo[3,4-d]pyrimidin-4-yl}-amine•3 TFA

The same procedure as described in example 1 is used, except that 4-diproylamino-piperidine is used instead of N-methylpiperazine and in step 1.1 2-chloraniline is used instead of o-toluidine. The title compound is obtained as an off-white solid.

HPLC t_{R}²=4.28 min; MS-ES+: (M+H)+=504. R_{f}**=0.56.

### Example 46

### (2-Chloro-phenyl)-{1-[4-((S)-3-dimethylamino-pyrrolidin-1-yl)-phenyl]-1H-pyrazolo[3,4-d]pyrimidin-4-yl}-amine•3 TFA

The same procedure as described in example 1 is used, except that (S)-3-dimethylamino-pyrrolidine is used instead of N-methylpiperazine and in step 1.1 2-chloraniline is used instead of o-toluidine. The title compound is obtained as an off-white solid. HPLC t_{R}²=3.33 min; MS-ES+: (M+H)+=434. R_{f}**=0.40.

### Example 47

### (2-Chloro-phenyl)-{1-[4-((R)-3-dimethylamino-pyrrolidin-1-yl)-phenyl]-1H-pyrazolo[3,4-d]pyrimidin-4-yl}-amine•3 TFA

The same procedure as described in example 1 is used, except that (R)-3-dimethylamino-pyrrolidine is used instead of N-methylpiperazine and in step 1.1 2-chloraniline is used instead of o-toluidine. The title compound is obtained as an off-white solid. HPLC t_{R}¹=2.51 min; MS-ES+: (M+H)+=434. R_{f}**=0.40.

### Example 48

### (2-Chloro-phenyl)-(1-{4-[4-(1-methyl-piperidin-4-yl)-piperazin-1-yl]-phenyl}-1H-pyrazolo[3,4-d]pyrimidin-4-yl)-amine•4 TFA

The same procedure as described in example 1 is used, except that 4-(1-methylpiperidin-4-yl)-piperazine is used instead of N-methylpiperazine and in step 1.1 2-chloraniline is used instead of o-toluidine. The title compound is obtained as an off-white solid. HPLC t_{R}¹=2.24 min; MS-ES+: (M+H)+=503. R_{f}**=0.18.

### Example 49

### (4-Fluoro-2-methyl-phenyl)-{1-[4-(4-pyrrolidin-1-yl-piperidin-1-yl)phenyl]-1H-pyrazolo[3,4-d]pyrimidin-4-yl}-amine•3 TFA

The same procedure as described in example 1 is used, except that 4-pyrrolidin-1-yl-piperidine is used instead of N-methylpiperazine and in step 1.1 2-methyl-4-fluoroaniline is used instead of o-toluidine. The title compound is obtained as an off-white solid. HPLC t_{R}¹=2.59 min; MS-ES+: (M+H)+=472. R_{f}**=0.37.

### Example 50

### (4-Fluoro-2-methyl-phenyl)-{1-[4-(4-methyl-piperazin-1-yl)-phenyl]-1H-pyrazolo[3,4-d]pyrimidin-4-yl}-amine•3 TFA

The same procedure as described in example 1 is used, except that in step 1.1 2-methyl-4-fluoroaniline is used instead of o-toluidine. The title compound is obtained as an off-white solid. HPLC t_{R}¹=2.13 min; MS-ES+: (M+H)+=418. R_{f}**=0.37.

### Example 51

### (4-Fluoro-2-methyl-phenyl)-(1-{4-[4-(4-methyl-piperazin-1-yl)-piperidin-1-yl]-phenyl}-1H-pyrazolo[3,4-d]pyrimidin-4-yl)-amine•4 TFA

The same procedure as described in example 1 is used, except that 4-(4-methylpiperazin-1-yl)-piperidine is used instead of N-methylpiperazine and in step 1.1 2-methyl-4-fluoroaniline is used instead of o-toluidine. The title compound is obtained as an off-white solid. HPLC t_{R}²=3.00 min; MS-ES+: (M+H)+=501. R_{f}**=0.29.

### Example 52

### {1-[4-(4-Diethylamino-piperidin-1-yl)-phenyl]-1H-pyrazolo[3,4-d]pyrimidin-4-yl}-(4-fluoro-2-methyl-phenyl)-amine•3 TFA

The same procedure as described in example 1 is used, except that 4-diethylaminopiperidine is used instead of N-methylpiperazine and in step 1.1 2-methyl-4-fluoroaniline is used instead of o-toluidine. The title compound is obtained as an off-white solid. HPLC t_{R}¹=2.63 min; MS-ES+: (M+H)+=474.

### Example 53

### {1-[4-(4-Dipropylamino-piperidin-1-yl)-phenyl]-1H-pyrazolo[3,4-d]pyrimidin-4-yl}-(4-fluoro-2-methyl-phenyl)-amine•3 TFA

The same procedure as described in example 1 is used, except that 4-dipropylamino-piperidine is used instead of N-methylpiperazine and in step 1.1 2-methyl-4-fluoroaniline is used instead of o-toluidine. The title compound is obtained as an off-white solid. HPLC t_{R}¹=2.93 min; MS-ES+: (M+H)+=502. R_{f}**=0.52.

### Example 54

### {1-[4-((R)-3-Dimethylamino-pyrrolidin-1-yl)-phenyl]-1H-pyrazolo[3,4-d]pyrimidin-4-yl}-(4-fluoro-2-methyl-phenyl)-amine•3 TFA

The same procedure as described in example 1 is used, except that (R)-3-dimethylamino-pyrrolidine is used instead of N-methylpiperazine and in step 1.1 2-methyl-4-fluoroaniline is used instead of o-toluidine. The title compound is obtained as an off-white solid. HPLC t_{R}¹=2.41 min; MS-ES+: (M+H)+=432. R_{f}**=0.34.

### Example 55

### {1-[4-((S)-3-Dimethylamino-pyrrolidin-1-yl)-phenyl]-1H-pyrazolo[3,4-d]pyrimidin-4-yl}-(4-fluoro-2-methyl-phenyl)-amine•3 TFA

The same procedure as described in example 1 is used, except that (S)-3-dimethylamino-pyrrolidine is used instead of N-methylpiperazine and in step 1.1 2-methyl-4-fluoroaniline is used instead of o-toluidine. The title compound is obtained as an off-white solid. HPLC t_{R}¹=2.41 min; MS-ES+: (M+H)+=432. R_{f}**=0.34.

### Example 56

### (4-Fluoro-2-methyl-phenyl)-(1-{4-[4-(1-methyl-piperidin-4-yl)-piperazin-1-yl]-phenyl}-1H-pyrazolo[3,4-d]pyrimidin-4-yl)-amine•4 TFA

The same procedure as described in example 1 is used, except that 4-(1-methylpiperidin-4-yl)-piperazine is used instead of N-methylpiperazine and in step 1.1 2-methyl-4-fluoroaniline is used instead of o-toluidine. The title compound is obtained as an off-white solid. HPLC t_{R}¹=2.17 min; MS-ES+: (M+H)+=501. R_{f}**=0.16.

### Example 57

### (4-Fluoro-2,6-dimethyl-phenyl)-{1-[4-(4-pyrrolidin-1-yl-piperidin-1-yl)-phenyl]-1H-pyrazolo[3,4-d]pyrimidin-4-yl}-amine•3 TFA

The same procedure as described in example 1 is used, except that 4-pyrrolidin-1-yl-piperidine is used instead of N-methylpiperazine and in step 1.1 2,6-dimethyl-4-fluoroaniline is used instead of o-toluidine. The title compound is obtained as an off-white solid. HPLC t_{R}¹=2.66 min; MS-ES+: (M+H)+=486. R_{f}**=0.34.

### Example 58

### (4-Fluoro-2,6-dimethyl-phenyl)-{1-[4-(4-methyl-piperazin-1-yl)-phenyl]-1H-Pyrazolo[3,4-d]pyrimidin-4-yl}-amine•3 TFA

The same procedure as described in example 1 is used, except that in step 1.1 2,6-dimethyl-4-fluoroaniline is used instead of o-toluidine. The title compound is obtained as an off-white solid. HPLC t_{R}¹=2.44 min; MS-ES+: (M+H)+=432.

### Example 59

### (4-Fluoro-2,6-dimethyl-phenyl)-(1-{4-[4-(4-methyl-piperazin-1-yl)-piperidin-1-yl]-phenyl}-1H-pyrazolo[3,4-d]pyrimidin-4-yl)-amine•4 TFA

The same procedure as described in example 1 is used, except that 4-(4-methylpiperazin-1-yl)-piperidine is used instead of N-methylpiperazine and in step 1.1 2,6-dimethyl-4-fluoroaniline is used instead of o-toluidine. The title compound is obtained as an off-white solid. HPLC t_{R}²=3.41 min; MS-ES+: (M+H)+=512.

### Example 60

### {1-[4-(4-Diethylamino-piperidin-1-yl)-phenyl]-1H-pyrazolo[3,4-d]pyrimidin-4-yl}-(4-fluoro-2,6-dimethyl-phenyl)-amine•3 TFA

The same procedure as described in example 1 is used, except that 4-diethylaminopiperidine is used instead of N-methylpiperazine and in step 1.1 2,6-dimethyl-4-fluoroaniline is used instead of o-toluidine. The title compound is obtained as an off-white solid. HPLC t_{R}^{1=2.73} min; MS-ES+: (M+H)+=488. R_{f}**=0.39.

### Example 61

### {1-[4-(4-Dipropylamino-piperidin-1-yl)-phenyl]-1H-pyrazolo[3,4-d]pyrimidin-4-yl}-(4-fluoro-2,6-dimethyl-phenyl)-amine•3 TFA

The same procedure as described in example 1 is used, except that 4-dipropylamino-piperidine is used instead of N-methylpiperazine and in step 1.1 2,6-dimethyl-4-fluoroaniline is used instead of o-toluidine. The title compound is obtained as an off-white solid. HPLC t_{R}²=4.43 min; MS-ES+: (M+H)+=516.

### Example 62

### {1-[4-((R)-3-Dimethylamino-pyrrolidin-1-yl)-phenyl]-1H-pyrazolo[3,4-d]pyrimidin-4-yl}-(4-fluoro-2,6-dimethyl-phenyl)-amine•3 TFA

The same procedure as described in example 1 is used, except that (R)-3-dimethylamino-pyrrolidine is used instead of N-methylpiperazine and in step 1.1 2,6-dimethyl-4-fluoroaniline is used instead of o-toluidine. The title compound is obtained as an off-white solid. HPLC t_{R}²=3.54 min; MS-ES+: (M+H)+=446.

### Example 63

### {1-[4-((S)-3-Dimethylamino-pyrrolidin-1-yl)-phenyl]-1H-pyrazolo[3,4-d]pyrimidin-4-yl}-(4-fluoro-2,6-dimethyl-phenyl)-amine•3 TFA

The same procedure as described in example 1 is used, except that (S)-3-dimethylamino-pyrrolidine is used instead of N-methylpiperazine and in step 1.1 2,6-dimethyl-4-fluoroaniline is used instead of o-toluidine. The title compound is obtained as an off-white solid. HPLC t_{R}²=3.52 min; MS-ES+: (M+H)+=446.

### Example 64

### (2-Chloro-4-Fluoro-phenyl)-{1-[4-(4-pyrrolidin-1-yl-piperidin-1-yl)phenyl]-1H-pyrazolo[3,4-d]pyrimidin-4-yl}-amine•3 TFA

The same procedure as described in example 1 is used, except that 4-pyrrolidin-1-yl-piperidine is used instead of N-methylpiperazine and in step 1.1 2-Chloro-4-fluoroaniline is used instead of o-toluidine. The title compound is obtained as an off-white solid. HPLC t_{R}¹=2.64 min; MS-ES+: (M+H)+=492. R_{f}**=0.25.

### Example 65

### (2-Chloro-4-fluoro-phenyl)-{1-[4-(4-methyl-piperazin-1-yl)-phenyl]-1H-pyrazolo[3,4-d]pyrimidin-4-yl}-amine•3 TFA

The same procedure as described in example 1 is used, except that in step 1.1 2-Chloro-4-fluoroaniline is used instead of o-toluidine. The title compound is obtained as an off-white solid. HPLC t_{R}²=3.25 min; MS-ES+: (M+H)+=438. R_{f}**=0.29.

### Example 66

### (2-Chloro-4-fluoro-phenyl)-(1-{4-[4-(4-methyl-piperazin-1-yl)-piperidin-1-yl]-phenyl}-1H-pyrazolo[3,4-d]pyrimidin-4-yl)-amine•4 TFA

The same procedure as described in example 1 is used, except that 4-(4-methylpiperazin-1-yl)-piperidine is used instead of N-methylpiperazine and in step 1.1 2-Chloro-4-fluoroaniline is used instead of o-toluidine. The title compound is obtained as an off-white solid. HPLC t_{R}¹=2.37 min; MS-ES+: (M+H)+=521. R_{f}**=0.20.

### Example 67

### (2-Chloro-4-fluoro-phenyl)-{1-[4-(4-diethylamino-piperidin-1-yl)-phenyl]-1H-pyrazolo[3,4-d]pyrimidin-4-yl}-amine•3 TFA

The same procedure as described in example 1 is used, except that 4-diethylaminopiperidine is used instead of N-methylpiperazine and in step 1.1 2-Chloro-4-fluoroaniline is used instead of o-toluidine. The title compound is obtained as an off-white solid. HPLC t_{R}¹=2.41 min; MS-ES+: (M+H)+=494. R_{f}**=0.27.

### Example 68

### (2-Chloro-4-fluoro-phenyl)-{1-[4-(4-dipropylamino-piperidin-1-yl)phenyl]-1H-pyrazolo[3,4-d]pyrimidin-4-yl}-amine•3 TFA

The same procedure as described in example 1 is used, except that 4-dipropylamino-piperidine is used instead of N-methylpiperazine and in step 1.1 2-Chloro-4-fluoroaniline is used instead of o-toluidine. The title compound is obtained as an off-white solid. HPLC t_{R}²=4.28 min; MS-ES+: (M+H)+=522.

### Example 69

### (2-Chloro-4-fluoro-phenyl)-{1-[4-((S)-3-dimethylamino-pyrrolidin-1-yl)phenyl]-1H-pyrazolo[3,4-d]pyrimidin-4-yl}-amine•3 TFA

The same procedure as described in example 1 is used, except that (S)-3-dimethylamino-pyrrolidine is used instead of N-methylpiperazine and in step 1.1 2-Chloro-4-fluoroaniline is used instead of o-toluidine. The title compound is obtained as an off-white solid. HPLC t_{R}²=3.56 min; MS-ES+: (M+H)+=452.

### Example 70

### (2-Chloro-4-fluoro-phenyl)-{1-[4-((R)-3-dimethylamino-pyrrolidin-1-yl)phenyl]-1H-pyrazolo[3,4-d]pyrimidin-4-yl}-amine•3 TFA

The same procedure as described in example 1 is used, except that (R)-3-dimethylamino-pyrrolidine is used instead of N-methylpiperazine and in step 1.1 2-Chloro-4-fluoroaniline is used instead of o-toluidine. The title compound is obtained as an off-white solid. HPLC t_{R}²=3.55 min; MS-ES+: (M+H)+=452.

### Example 71

### (2-Chloro-4-fluoro-phenyl)-(1-{4-[4-(1-methyl-piperidin-4-yl)-piperazin-1-yl]-phenyl}-1H-pyrazolo[3,4-d]pyrimidin-4-yl)-amine•4 TFA

The same procedure as described in example 1 is used, except that 4-(1-methylpiperidin-4-yl)-piperazine is used instead of N-methylpiperazine and in step 1.1 2-Chloro-4-fluoroaniline is used instead of o-toluidine. The title compound is obtained as an off-white solid. HPLC t_{R}¹=1.94 min; MS-ES+: (M+H)+=521. R_{f}**=0.27.

### Example 72

### N,N-Dimethyl-N′-[4-(4-o-tolylamino-pyrazolo[3,4-d]pyrimidin-1-yl)phenyl]-ethane-1,2-diamine•3 TFA

The same procedure as described in example 1 is used, except that N,N-dimethylethane-1,2-diamine is used instead of N-methylpiperazine. The title compound is obtained as an off-white solid. HPLC t_{R}¹=1.87 min; MS-ES+: (M+H)+=416.

### Example 73

### (1-{3-[4-(4-Methyl-piperazin-1-yl)-piperidin-1-yl]-phenyl}-1H-pyrazolo[3,4-d]pyrimidin-4-yl)-o-tolyl-amine

The same procedure as described in example 1 is used, except that 1-methyl-4-(piperidin-4-yl)-piperazine is used instead of N-methylpiperazine and in step 1.5 3-bromophenylhydrazine is used instead of 4-bromophenylhydrazine. The crude product was purified by reverse phase chromatography. The title compound is obtained as free base as an off-white solid.

HPLC: t_{R}³=7.260 min.; MS-ES: (M+H)+=483; TLC: R_{f}=0.52 (CH₂Cl₂/MeOH=4/1)

### Example 74

### (4-Fluoro-2-methyl-phenyl)-(1-{3-[4-(4-methyl-piperazin-1-yl) -piperidin-1-yl]-phenyl}-1H-pyrazolo[3,4-d]pyrimidin-4-yl)-amine

The same procedure as described in example 1 is used, except that 1-methyl-4-(piperidin-4-yl)-piperazine is used instead of N-methylpiperazine, in step 1.1 4-fluoro-2-methylaniline is used instead of o-toluidine and in step 1.5 3-bromophenylhydrazine is used instead of 4-bromophenylhydrazine. The crude product was purified by reverse phase chromatography. The title compound is obtained as free base as an off-white solid.

HPLC: t_{R}³=7.202 min.; MS-ES: (M+H)+=501; TLC: R_{f}=0.60 (CH₂Cl₂/MeOH=4/1)

### Example 75

### (1-{4-[4-(4-Methyl-piperazin-1-yl)-piperidin-1-yl]-phenyl}-6-phenyl-1H-pyrazolo[3,4-d]pyrimidin-4-yl)-o-tolyl-amine

The same procedure as described in example 1 is used, except that 1-methyl-4-(piperidin-4-yl)-piperazine is used instead of N-methylpiperazine and instead of step 1.3 step 2.1 (example 2) with benzoic acid instead of 4-(dimethylamino)butyric acid hydrochloride salt is used. The crude product was purified by reverse phase chromatography. The title compound is obtained as free base as an off-white solid. HPLC: t_{R}³=8.816 min.; MS-ES: (M+H)+=559; TLC: R_{f}=0.51 (CH₂Cl₂/MeOH=9/1)

### Example 76

### {1-[4-(4-Diethylamino-piperidin-1-yl)-phenyl]-6-Phenyl-1H-pyrazolo[3,4-d]pyrimidin-4-yl}-o-tolyl-amine

The same procedure as described in example 1 is used, except that 4-diethylaminopiperidine is used instead of N-methylpiperazine and instead of step 1.3 step 2.1 (example 2) with benzoic acid instead of 4-(dimethylamino)butyric acid hydrochloride salt is used. The crude product was purified by reverse phase chromatography. The title compound is obtained as free base as an off-white solid.

HPLC: t_{R}³=10.054 min.; MS-ES: (M+H)+=532; TLC: R_{f} 0.33 (CH₂Cl₂/MeOH=9/1)

### Example 77

### (1-{4-[4-(4-Methyl-piperazin-1-yl)-piperidin-1-yl]-phenyl}-6-pyridin-2-yl-1H-pyrazolo[3,4-d]pyrimidin-4-yl)-o-tolyl-amine

The same procedure as described in example 1 is used, except that 1-methyl-4-(piperidin-4-yl)-piperazine is used instead of N-methylpiperazine and instead of step 1.3 step 2.1 (example 2) with picolinic acid instead of 4-(dimethylamino)butyric acid hydrochloride salt is used. The crude product was purified by reverse phase chromatography. The title compound is obtained as free base as an off-white solid. HPLC: t_{R}³=7.147 min.; MS-ES: (M+H)+=560; TLC: R_{f}=0.16 (CH₂Cl₂/MeOH=4/1)

### Example 78

### (1-{4-[4-(4-Methyl-piperazin-1-yl)-piperidin-1-yl]-phenyl}-6-pyridin-3-yl-1H-pyrazolo[3,4-d]pyrimidin-4-yl)-o-tolyl-amine

The same procedure as described in example 1 is used, except that 1-methyl-4-(piperidin-4-yl)-piperazine is used instead of N-methylpiperazine and instead of step 1.3 step 2.1 (example 2) with nicotinic acid instead of 4-(dimethylamino)butyric acid hydrochloride salt is used. The crude product was purified by reverse phase chromatography. The title compound is obtained as free base as an off-white solid. HPLC: t_{R}³=7.236 min.; MS-ES: (M+H)+=560; TLC: R_{f}=0.57 (CH₂Cl₂/MeOH=4/1)

### Example 79

### (1-{4-[4-(4-Methyl-piperazin-1-yl)-piperidin-1-yl]-phenyl}-6-pyridin-4-yl-1H-pyrazolo[3,4-d]pyrimidin-4-yl)-o-tolyl-amine

The same procedure as described in example 1 is used, except that 1-methyl-4-(piperidin-4-yl)-piperazine is used instead of N-methylpiperazine and instead of step 1.3 step 2.1 (example 2) with isonicotinic acid instead of 4-(dimethylamino)butyric acid hydrochloride salt is used. The crude product was purified by reverse phase chromatography. The title compound is obtained as free base as an off-white solid. HPLC: t_{R}³=7.298 min.; MS-ES: (M+H)+=560; TLC: R_{f}=0.48 (CH₂Cl₂/MeOH=4/1)

### Example 80

### {1-[4-(4-Diethylamino-piperidin-1-yl)-phenyl]-6-pyridin-4-yl-1H-pyrazolo[3,4-d]pyrimidin-4-yl}-o-tolyl-amine

The same procedure as described in example 1 is used, except that 4-diethylaminopiperidine is used instead of N-methylpiperazine and instead of step 1.3 step 2.1 (example 2) with isonicotinic acid instead of 4-(dimethylamino)butyric acid hydrochloride salt is used. The crude product was purified by reverse phase chromatography. The title compound is obtained as free base as an off-white solid. HPLC: t_{R}³=7.297 min.; MS-ES: (M+H)+=533; TLC: R_{f}=0.60 (CH₂Cl₂/MeOH=4/1)

The following Examples is prepared in analogy to the preceding Examples:

| Example | Formula |
|---|---|
| 81 | 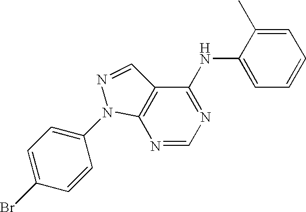 |

### Example 82

### Soft Capsules

5000 soft gelatin capsules, each comprising as active ingredient 0.05 g of any one of the compounds of formula I mentioned in any one of the preceding Examples, are prepared as follows:

Composition

| | | |
|---|---|---|
| Active ingredient | 250 | g |
| Lauroglycol | 2 | liters |

Preparation process: The pulverized active ingredient is suspended in Lauroglykol* (propylene glycol laurate, Gattefossé S. A., Saint Priest, France) and ground in a wet pulverizer to produce a particle size of about 1 to 3 μm. 0.419 g portions of the mixture are then introduced into soft gelatin capsules using a capsule-filling machine.

### Example 83

### Tablets Comprising Compounds of the Formula I

Tablets, comprising, as active ingredient, 100 mg of any one of the compounds of formula I in any one of the preceding Examples are prepared with the following composition, following standard procedures:

Composition

| | | |
|---|---|---|
| Active Ingredient | 100 | mg |
| crystalline lactose | 240 | mg |
| Avicel | 80 | mg |
| PVPPXL | 20 | mg |
| Aerosil | 2 | mg |
| magnesium stearate | 5 | mg |
| | 447 | mg |

Manufacture: The active ingredient is mixed with the carrier materials and compressed by means of a tabletting machine (Korsch EKO, stamp diameter 10 mm).

Avicel® is microcrystalline cellulose (FMC, Philadelphia, USA). PVPPXL is polyvinylpolypyrrolidone, cross-linked (BASF, Germany). Aerosil® is silicon dioxide (Degussa, Germany).

### Example 84

### Inhibition of EphB4 Kinase Activity

Using the test system described above in the general description, inter alia the compounds of Examples 21, 23 and 27 are tested for their ability to inhibit EphB4 kinase. The following IC₅₀ values (μmol/l) are found:

TABLE
“Inhibition of EphB4 kinase”

| Compound of Example | IC₅₀ (μmol/l) |
|---|---|
| 21 | 0.16 |
| 23 | 0.5 |
| 27 | 0.4 |

1. A compound of the formula I, 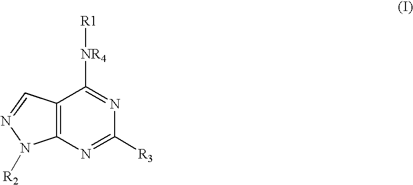
wherein
R₁ is a moiety of the formula Ib 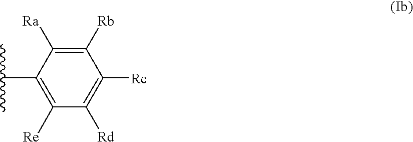
wherein Ra is methyl, ethyl, methoxy, halo or trifluoromethyl;
Re is hydrogen, methyl, ethyl, methoxy, halo or trifluoromethyl, and
Rb, Rc and Rd are independently selected from hydrogen and phenyl substitutents;
R₂ is unsubstituted or substituted aryl;
R₃ is hydrogen, unsubstituted or substituted alkyl, unsubstituted or substituted aryl or unsubstituted or substituted heterocyclyl; and
R₄ is hydrogen or unsubstituted or substituted alkyl;
or a pharmaceutically acceptable salt thereof where one or more salt-forming groups are present, for use in the diagnostic or therapeutic treatment of a warm-blooded animal.

2. A compound of the formula I, or a pharmaceutically acceptable salt thereof, according to claim 1 for use in the treatment of a disease that depends on inadequate activity of a protein kinase, preferably a protein tyrosine kinase, especially one or more of c-Abl, c-Src and/or preferably an Ephrin receptor kinase, more especially EphB4 kinase; and/or one or more altered or mutated forms of any one or more of these, e.g. those forms that result in conversion of the respective proto-oncogene into an oncogene, such as constitutively activated Bcr-Abl or v-Src.

3. The use of a compound of the formula I, or a pharmaceutically acceptable salt thereof, as defined in claim 1 in the preparation of a pharmaceutical formulation for the treatment of a disease or disorder that depends on inadequate activity of a protein kinase, preferably a protein tyrosine kinase, especially one or more of c-Abl, c-Src and/or especially Ephrin receptor kinase, more especially EphB4 kinase; and/or one or more altered or mutated forms of any one or more of these, e.g. those forms that result in conversion of the respective proto-oncogene into an oncogene, such as constitutively activated Bcr-Abl or v-Src.

4. A compound of the formula I, 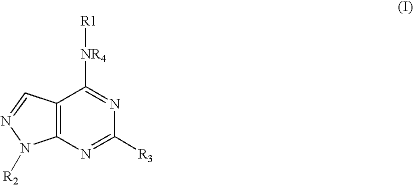
wherein
R₁ is a moiety of the formula Ib 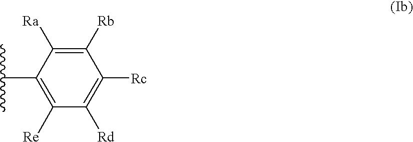
wherein Ra is methyl, ethyl, methoxy, halo or trifluoromethyl;
Re is hydrogen, methyl, ethyl, methoxy, halo or trifluoromethyl, and
Rb, Rc and Rd are independently selected from hydrogen, unsubstituted or substituted alkyl, unsubstituted or substituted alkenyl, unsubstituted or substituted alkynyl, unsubstituted or substituted aryl, unsubstituted or substituted heterocyclyl, hydroxy, esterified or etherified hydroxy, unsubstituted, mono- or disubstituted amino wherein the substitutents are independently selected from unsubstituted or substituted alkyl and unsubstituted or substituted aryl; halo, nitro, cyano, mercapto, substituted mercapto, sulfo and substituted sulfonyl wherein the substituents are selected from unsubstituted or substituted alkyl and unsubstituted or substituted aryl;
R₂ is unsubstituted or substituted aryl;
R₃ is hydrogen, unsubstituted or substituted alkyl, unsubstituted or substituted aryl or unsubstituted or substituted heterocyclyl; and
R₄ is hydrogen or unsubstituted or substituted alkyl,
with the proviso that if R₂ is 4-methoxyphenyl, R₃ is hydrogen and R₄ is hydrogen, then R₁ is other than 5-fluoro-2-methylphenyl and 2-methylphenyl; and with the proviso that R₁ is other than unsubstituted or substituted 3-nitrophenyl;
or a salt thereof.

5. A compound of the formula I according to claim 4, wherein
R₁ is a moiety of the formula Ib as shown in claim 4
wherein Ra is methyl, ethyl, methoxy, halo or trifluoromethyl,
Re is hydrogen, methyl, ethyl, methoxy, halo or trifluoromethyl, and
Rb, Rc and Rd are independently selected from hydrogen (preferred), C₁-C₇-alkyl, C₂-C₇-alkenyl, C₂-C₇-alkynyl, hydroxy, C₁-C₇-alkoxy, amino, N-mono- or N,N-di-(C₁-C₇-alkyl)amino; halo (preferred), nitro and cyano;
R₂ is substituted phenyl wherein the substituents are one or more, preferably one or two, especially one, substituents independently selected from the group consisting of
a 3- to 8-membered heterocyclic ring, preferably bound via a ring nitrogen atom, containing, in addition to one or more carbon ring atoms, one to four nitrogen where instead of an H in —NH—C₁-C₇-alkyl may be present, oxygen or sulfur atoms (e.g. azepinyl, especially azepino, diazepinyl (such as 1,4-diazepinyl), especially diazepino, (especially N-) C₁-C₇-alkyl-diazepinyl or preferably N—C₁-C₇-alkyldiazepino, piperidinyl, especially piperidino, morpholinyl, especially morpholino, thiomorpholinyl, especially thiomorpholino, piperazinyl. especially piperazino, (especially N-) C₁-C₇-alkyl-piperazinyl, especially N—C₁-C₇-alkyl-piperazino, pyrrolidinyl, especially pyrrolidino, imidazolidinyl, especially imidazolidino, (especially N-) C₁-C₇-alkyl-imidazolidinyl, preferably N—C₁-C₇-alkyl-imidazolidino, pyrazolidinyl, especially pyrazolidino, (especially N-) C₁-C₇-alkylpyrazolidinyl, preferably C₁-C₇-alkylpyrazolidino, azetidinyl, especially azetidino, or aziridinyl, especially aziridino) which ring is unsubstituted or substituted by either
(i) a 3- to 8-membered heterocyclic ring, preferably bound via a ring carbon or nitrogen atom, containing, in addition to one or more carbon ring atoms, one to four nitrogen where instead of an H in NH C₁-C₇-alkyl may be present, oxygen or sulfur atoms (e.g. azepinyl, diazepinyl (such as 1,4-diazepinyl), (especially N-) C₁-C₇-alkyl-diazepinyl, piperidinyl, morpholinyl, thiomorpholinyl, piperazinyl, (especially N-) C₁-C₇-alkyl-piperazinyl, pyrrolidinyl, imidazolidinyl, (especially N-) C₁-C₇-alkyl-imidazolidinyl, pyrazolidinyl, (especially N-) C₁-C₇-alkylpyrazolidinyl, azetidinyl or aziridinyl)
(ii) amino-C₁-C₇-alkyl or by N-mono or N,N-disubstituted amino-C₁-C₇-alkyl, wherein the amino substituents are independently selected from C₁-C₇-alkyl, C₁-C₇-alkanoyl, phenyl and phenyl-C₁-C₇-alkyl, e.g. N,N-di-(C₁-C₇-alkyl)amino-C₁-C₇-alkyl, such as N,N-dimethylamino-C₁-C₇-alkyl, or
(iii) hydroxy-C₁-C₇-alkyl, e.g. hydroxymethyl, C₁-C₇-alkoxy-C₁-C₇-alkyl, (C₁-C₇-alkoxy)-C₁-C₇-alkoxy-C₁-C₇-alkyl, C₁-C₇-alkanoyl-C₁-C₇-alkyl, phenoxy-C₁-C₇-alkyl, phenyl-C₁-C₇-alkoxy-lower alkyl, such as benzyloxy-C₁-C₇-alkyl, C₁-C₇-alkoxy-carbonyloxy-C₁-C₇-alkyl, such as tert-butoxycarbonyloxy-C₁-C₇-alkyl or phenyl-C₁-C₇-alkoxycarbonyloxy-C₁-C₇-alkyl, such as benzyloxycarbonyloxy-C₁-C₇-alkyl;
N-mono- or N,N-di-(C₁-C₇-alkyl)-amino-C₁-C₇-alkyl-amino and halo;
R₃ is hydrogen, C₁-C₇-alkyl or amino-, N-mono- or N,N-di-(C₁-C₇-alkyl)-amino-C₁-C₇-alkyl, phenyl or pyridyl; and
R₄ is hydrogen,
or a salt thereof.

6. A compound of the formula I, wherein
R₁ is a moiety of the formula Ib as shown above wherein
Ra is methyl, ethyl, methoxy, halo or trifluoromethyl
Re is hydrogen, methyl, ethyl, methoxy, halo or trifluoromethyl
and Rb, Rc and Rd are independently selected from hydrogen and halo;
R₂ is phenyl or phenyl that is substituted, especially in the 3- or 4-position, by halo, especially bromo, or preferably 4-(4-methyl-piperazin-1-yl), 4-morpholin-4-yl-, 4-(4-pyrrolidin-1-yl-piperidin-1-yl), 4-(4-morpholin-4-yl-piperidin-1-yl), 4-[4-(4-methyl-piperazin-1-yl)-piperidin-1-yl, 3-[4-(4-methylpiperazin-1-yl)-piperidin-1-yl, 4-(4-diethylamino-piperidin-1-yl), 4-(4-dipropylamino-piperidin-1-yl), 4-((R,S)-, 4-((R)- or 4-((S)-3-dimethylamino-pyrrolidin-1-yl), 4-(4-methyl-[1,4]-diazepan-1-yl), 4-[4-(1-methyl-piperidin-4-yl)-piperazin-1-yl], 3-(4-methyl-piperazin-1-yl), or 2-(N,N-dimethylamino)ethylamino
R₃ is hydrogen, C₁-C₇-alkyl, especially methyl, or amino-, N-mono- or N,N-di-(C₁-C₇-alkyl)-amino-C₁-C₇-alkyl, especially (3-dimethylamino-propyl, phenyl or pyridyl, and
R₄ is hydrogen,
or a (preferably pharmaceutically acceptable) salt thereof.

7. A compound of the formula I according to claim 4, selected from the group consisting of
{1-[4-(4-methyl-piperazin-1-yl)-phenyl]-1H-pyrazolo[3,4-d]pyrimidin-4-yl}-o-tolyl-amine,
[6-(3-dimethylamino-propyl)-1-phenyl-1H-pyrazolo[3,4-d]pyrimidin-4-yl]-o-tolyl-amine,
[1-(4-morpholin-4-yl-phenyl)-1H-pyrazolo[3,4-d]pyrimidin-4-yl]-o-tolyl-amine,
(2,6-dimethyl-phenyl)-{1-[4-(4-methyl-piperazin-1-yl)-phenyl]-1H-pyrazolo[3,4-d]pyrimidin-4-yl}-amine,
{1-[3-(4-methyl-piperazin-1-yl)-phenyl]-1H-pyrazolo[3,4-d]pyrimidin-4-yl}-o-tolyl-amine,
(2,6-dimethyl-phenyl)-{1-[3-(4-methyl-piperazin-1-yl)-phenyl]-1H-pyrazolo[3,4-d]pyrimidin-4-yl}-amine,
{1-[4-(4-pyrrolidin-1-yl-piperidin-1-yl)-phenyl]-1H-pyrazolo[3,4-d]pyrimidin-4-yl}-o-tolyl-amine,
(2,6-dimethyl-phenyl)-{1-[4-(4-pyrrolidin-1-yl-piperidin-1-yl)-phenyl]-1H-pyrazolo[3,4-d]pyrimidin-
{1-[4-(4-morpholin-4-yl-piperidin-1-yl)-phenyl]-1H-pyrazolo[3,4-d]pyrimidin-4-yl}-o-tolyl-amine,
(2,6-dimethyl-phenyl)-{1-[4-(4-morpholin-4-yl-piperidin-1-yl)-phenyl]-1H-pyrazolo[3,4-d]pyrimidin-4-yl}-amine,
(1-{4-[4-(4-methyl-piperazin-1-yl)-piperidin-1-yl]-phenyl}-1H-pyrazolo[3,4-d]pyrimidin-4-yl)-o-tolylamine,
(2,6-dimethyl-phenyl)-(1-{4-[4-(4-methyl-piperazin-1-yl)-piperidin-1-yl]-phenyl}-1H-pyrazolo[3,4-d]pyrimidin-4-yl)-amine,
{1-[4-(4-diethylamino-piperidin-1-yl)-phenyl]-1H-pyrazolo[3,4-d]pyrimidin-4-yl}-o-tolyl-amine,
{1-[4-(4-dipropylamino-piperidin-1-yl)-phenyl]-1H-pyrazolo[3,4-d]pyrimidin-4-yl}-o-tolyl-amine,
{1-[4-((R)-3-dimethylamino-pyrrolidin-1-yl)-phenyl]-1H-pyrazolo[3,4-d]pyrimidin-4-yl}-o-tolylamine,
{1-[4-((S)-3-dimethylamino-pyrrolidin-1-yl)-phenyl]-1H-pyrazolo[3,4-d]pyrimidin-4-yl}-o-tolylamine,
{1-[4-(4-methyl-[1,4]diazepan-1-yl)-phenyl]-1H-pyrazolo[3,4-d]pyrimidin-4-yl}-o-tolyl-amine,
(1-{4-[4-(1-methyl-piperidin-4-yl)-piperazin-1-yl]-phenyl}-1H-pyrazolo[3,4-d]pyrimidin-4-yl)-o-tolylamine,
{6-methyl-1-[4-(4-methyl-piperazin-1-yl)-phenyl]-1H-pyrazolo[3,4-d]pyrimidin-4-yl}-o-tolyl-amine,
{6-methyl-1-[3-(4-methyl-piperazin-1-yl)-phenyl]-1H-pyrazolo[3,4-d]pyrimidin-4-yl}-o-tolyl-amine,
(2,6-dimethyl-phenyl)-[1-(4-methoxy-phenyl)-1H-pyrazolo[3,4-d]pyrimidin-4-yl]-amine,
[1-(3-methoxy-phenyl)-1H-pyrazolo[3,4-d]pyrimidin-4-yl]-o-tolyl-amine,
(2,6-dimethyl-phenyl)-{1-(3-methoxy-phenyl)-1H-pyrazolo[3,4-d]pyrimidin-4-yl}-amine,
{6-methyl-1-phenyl-1H-pyrazolo[3,4-d]pyrimidin-4-yl}-o-tolyl-amine,
[6-(3-dimethylamino-propyl)-1-phenyl-1H-pyrazolo[3,4-d]pyrimidin-4-yl]-(2,6-dimethyl-phenyl)amine,
{6-(3-dimethylamino-propyl)-1-phenyl-1H-pyrazolo[3,4-d]pyrimidin-4-yl}-(2-chlor-phenyl)-amine,
{1-[4-bromophenyl]-1H-pyrazolo[3,4-d]pyrimidin-4-yl}-o-tolyl-amine,
{1-[4-(4-diethylamino-piperidin-1-yl)-phenyl]-1H-pyrazolo[3,4-d]pyrimidin-4-yl}-(2,6-dimethylphenyl)-amine,
(2,6-Dimethyl-phenyl)-{1-[4-(4-dipropylamino-piperidin-1-yl)-phenyl]-1H-pyrazolo[3,4-d]pyrimidin-4-yl}-amine,
(2,6-Dimethyl-phenyl)-{1-[4-((R)-3-dimethylamino-pyrrolidin-1-yl)-phenyl]-1H-pyrazolo[3,4-d]pyrimidin-4-yl}-amine,
(2,6-Dimethyl-phenyl)-(1-{4-[4-(1-methyl-piperidin-4-yl)-piperazin-1-yl]-phenyl}-1H-pyrazolo[3,4-d]pyrimidin-4-yl)-amine,
(5-Fluoro-2-methyl-phenyl)-{1-[4-(4-pyrrolidin-1-yl-piperidin-1-yl)-phenyl]-1H-pyrazolo[3,4-d]pyrimidin-4-yl}-amine,
(5-Fluoro-2-methyl-phenyl)-{1-[4-(4-methyl-piperazin-1-yl)-phenyl]-1H-pyrazolo[3,4-d]pyrimidin-4-yl}-amine,
(5-Fluoro-2-methyl-phenyl)-(1-{4-[4-(4-methyl-piperazin-1-yl)-piperidin-1-yl]-phenyl}-1H-pyrazolo[3,4-d]pyrimidin-4-yl)-amine,
{1-[4-(4-Diethylamino-piperidin-1-yl)-phenyl]-1H-pyrazolo[3,4-d]pyrimidin-4-yl}-(5-fluoro-2-methyl-phenyl)-amine,
{1-[4-(4-Diproylamino-piperidin-1-yl)-phenyl]-1H-pyrazolo[3,4-d]pyrimidin-4-yl}-(5-fluoro-2-methyl-phenyl)-amine,
{1-[4-((R)-3-Dimethylamino-pyrrolidin-1-yl)-phenyl]-1H-pyrazolo[3,4-d]pyrimidin-4-yl}-(5-fluoro-2-methyl-phenyl)-amine,
{1-[4-((S)-3-Dimethylamino-pyrrolidin-1-yl)-phenyl]-1H-pyrazolo[3,4-d]pyrimidin-4-yl}-(5-fluoro-2-methyl-phenyl)-amine,
(5-Fluoro-2-methyl-phenyl)-(1-{4-[4-(1-methyl-piperidin-4-yl)-piperazin-1-yl]-phenyl}-1H-pyrazolo[3,4-d]pyrimidin-4-yl)-amine,
(2-Chloro-phenyl)-{1-[4-(4-pyrrolidin-1-yl-piperidin-1-yl)-phenyl]-1H-pyrazolo[3,4-d]pyrimidin-4-yl}-amine,
(2-Chloro-phenyl)-{1-[4-(4-methyl-piperazin-1-yl)-phenyl]-1H-pyrazolo[3,4-d]pyrimidin-4-yl}-amine,
(2-Chloro-phenyl)-(1-{4-[4-(4-methyl-piperazin-1-yl)-piperidin-1-yl]-phenyl}-1H-pyrazolo[3,4-d]pyrimidin-4-yl)-amine,
(2-Chloro-phenyl)-{1-[4-(4-diethylamino-piperidin-1-yl)-phenyl]-1H-pyrazolo[3,4-d]pyrimidin-4-yl}-amine,
(2-Chloro-phenyl)-{1-[4-(4-diproylamino-piperidin-1-yl)-phenyl]-1H-pyrazolo[3,4-d]pyrimidin-4-yl}-amine,
(2-Chloro-phenyl)-{1-[4-((S)-3-dimethylamino-pyrrolidin-1-yl)-phenyl]-1H-pyrazolo[3,4-d]pyrimidin-4-yl}-amine,
(2-Chloro-phenyl)-{1-[4-((R)-3-dimethylamino-pyrrolidin-1-yl)-phenyl]-1H-pyrazolo[3,4-d]pyrimidin-4-yl}-amine,
(2-Chloro-phenyl)-(1-{4-[4-(1-methyl-piperidin-4-yl)-piperazin-1-yl]-phenyl}-1H-pyrazolo[3,4-d]pyrimidin-4-yl)-amine,
(4-Fluoro-2-methyl-phenyl)-{1-[4-(4-pyrrolidin-1-yl-piperidin-1-yl)-phenyl]-1H-pyrazolo[3,4-d]pyrimidin-4-yl}-amine,
(4-Fluoro-2-methyl-phenyl)-{1-[4-(4-methyl-piperazin-1-yl)-phenyl]-1H-pyrazolo[3,4-d]pyrimidin-4-yl}-amine,
(4-Fluoro-2-methyl-phenyl)-(1-{4-[4-(4-methyl-piperazin-1-yl)-piperidin-1-yl]-phenyl}-1H-pyrazolo[3,4-d]pyrimidin-4-yl)-amine,
{1-[4-(4-Diethylamino-piperidin-1-yl)-phenyl]-1H-pyrazolo[3,4-d]pyrimidin-4-yl}-(4-fluoro-2-methyl-phenyl)-amine,
{1-[4-(4-Dipropylamino-piperidin-1-yl)-phenyl]-1H-pyrazolo[3,4-d]pyrimidin-4-yl}-(4-fluoro-2-methyl-phenyl)-amine,
{1-[4-((R)-3-Dimethylamino-pyrrolidin-1-yl)-phenyl]-1H-pyrazolo[3,4-d]pyrimidin-4-yl}-(4-fluoro-2-methyl-phenyl)-amine,
{1-[4-((S)-3-Dimethylamino-pyrrolidin-1-yl)-phenyl]-1H-pyrazolo[3,4-d]pyrimidin-4-yl}-(4-fluoro-2-methyl-phenyl)-amine,
(4-Fluoro-2-methyl-phenyl)-(1-{4-[4-(1-methyl-piperidin-4-yl)-piperazin-1-yl]-phenyl}-1H-pyrazolo[3,4-d]pyrimidin-4-yl)-amine,
(4-Fluoro-2,6-dimethyl-phenyl)-{1-[4-(4-pyrrolidin-1-yl-piperidin-1-yl)-phenyl]-1H-pyrazolo[3,4-d]pyrimidin-4-yl}-amine,
(4-Fluoro-2,6-dimethyl-phenyl)-{1-[4-(4-methyl-piperazin-1-yl)-phenyl]-1H-pyrazolo[3,4-d]pyrimidin-4-yl}-amine,
(4-Fluoro-2,6-dimethyl-phenyl)-(1-{4-[4-(4-methyl-piperazin-1-yl)-piperidin-1-yl]-phenyl}-1H-pyrazolo[3,4-d]pyrimidin-4-yl)-amine,
{1-[4-(4-Diethylamino-piperidin-1-yl)-phenyl]-1H-pyrazolo[3,4-d]pyrimidin-4-yl}-(4-fluoro-2,6-dimethyl-phenyl)-amine,
{1-[4-(4-Dipropylamino-piperidin-1-yl)-phenyl]-1H-pyrazolo[3,4-d]pyrimidin-4-yl}-(4-fluoro-2,6-dimethyl-phenyl)-amine,
{1-[4-((R)-3-Dimethylamino-pyrrolidin-1-yl)-phenyl]-1H-pyrazolo[3,4-d]pyrimidin-4-yl}-(4-fluoro-2,6-dimethyl-phenyl)-amine,
{1-[4-((S)-3-Dimethylamino-pyrrolidin-1-yl)-phenyl]-1H-pyrazolo[3,4-d]pyrimidin-4-yl}-(4-fluoro-2,6-dimethyl-phenyl)-amine,
(2-Chloro-4-Fluoro-phenyl)-{1-[4-(4-pyrrolidin-1-yl-piperidin-1-yl)-phenyl]-1H-pyrazolo[3,4-d]pyrimidin-4-yl}-amine,
(2-Chloro-4-fluoro-phenyl)-{1-[4-(4-methyl-piperazin-1-yl)-phenyl]-1H-pyrazolo[3,4-d]pyrimidin-4-yl}-amine,
(2-Chloro-4-fluoro-phenyl)-(1-{4-[4-(4-methyl-piperazin-1-yl)-piperidin-1-yl]-phenyl}-1H-pyrazolo[3,4-d]pyrimidin-4-yl)-amine,
(2-Chloro-4-fluoro-phenyl)-{1-[4-(4-diethylamino-piperidin-1-yl)-phenyl]-1H-pyrazolo[3,4-d]pyrimidin-4-yl}-amine,
(2-Chloro-4-fluoro-phenyl)-{1-[4-(4-dipropylamino-piperidin-1-yl)-phenyl]-1H-pyrazolo[3,4-d]pyrimidin-4-yl}-amine,
(2-Chloro-4-fluoro-phenyl)-{1-[4-((S)-3-dimethylamino-pyrrolidin-1-yl)-phenyl]-1H-pyrazolo[3,4-d]pyrimidin-4-yl}-amine,
(2-Chloro-4-fluoro-phenyl)-{1-[4-((R)-3-dimethylamino-pyrrolidin-1-yl)-phenyl]-1H-pyrazolo[3,4-d]pyrimidin-4-yl}-amine,
(2-Chloro-4-fluoro-phenyl)-(1-{4-[4-(1-methyl-piperidin-4-yl)-piperazin-1-yl]-phenyl}-1H-pyrazolo[3,4-d]pyrimidin-4-yl)-amine,
N,N-Dimethyl-N′-[4-(4-o-tolylamino-pyrazolo[3,4-d]pyrimidin-1-yl)-phenyl]-ethane-1,2-diamine,
(1-{3-[4-(4-Methyl-piperazin-1-yl)-piperidin-1-yl]-phenyl}-1H-pyrazolo[3,4-d]pyrimidin-4-yl)-o-tolylamine,
(4-Fluoro-2-methyl-phenyl)-(1-{3-[4-(4-methyl-piperazin-1-yl)-piperidin-1-yl]-phenyl}-1H-pyrazolo[3,4-d]pyrimidin-4-yl)-amine,
(1-{4-[4-(4-Methyl-piperazin-1-yl)-piperidin-1-yl]-phenyl}-6-phenyl-1H-pyrazolo[3,4-d]pyrimidin-4-yl)-o-tolyl-amine,
{1-[4-(4-Diethylamino-piperidin-1-yl)-phenyl]-6-phenyl-1H-pyrazolo[3,4-d]pyrimidin-4-yl}-o-tolylamine,
(1-{4-[4-(4-Methyl-piperazin-1-yl)-piperidin-1-yl]-phenyl}-6-pyridin-2-yl-1H-pyrazolo[3,4-d]pyrimidin-4-yl)-o-tolyl-amine,
(1-{4-[4-(4-Methyl-piperazin-1-yl)-piperidin-1-yl]-phenyl}-6-pyridin-3-yl-1H-pyrazolo[3,4-d]pyrimidin-4-yl)-o-tolyl-amine,
(1-{4-[4-(4-Methyl-piperazin-1-yl)-piperidin-1-yl]-phenyl}-6-pyridin-4-yl-1H-pyrazolo[3,4-d]pyrimidin-4-yl)-o-tolyl-amine, and
{1-[4-(4-Diethylamino-piperidin-1-yl)-phenyl]-6-pyridin-4-yl-1H-pyrazolo[3,4-d]pyrimidin-4-yl}-O— tolyl-amine or a salt thereof.

8. A compound of the formula I as shown in claim 1, selected from the group consisting of [1-(4-methoxy-phenyl)-1H-pyrazolo[3,4-d]pyrimidin-4-yl]-o-tolyl-amine and (5-fluoro-2-methyl-phenyl)-[1-(4-methoxy-phenyl)-1H-pyrazolo[3,4-d]pyrimidin-4-yl]-amine, or a pharmaceutically acceptable salt thereof, for use in the diagnostic or therapeutic treatment of a warm-blooded animal.

9. The use of a compound of the formula I given in claim 1 selected from the group consisting of
[1-(4-methoxy-phenyl)-1H-pyrazolo[3,4-d]pyrimidin-4-yl]-o-tolyl-amine,
(5-fluoro-2-methyl-phenyl)-[1-(4-methoxy-phenyl)-1H-pyrazolo[3,4-d]pyrimidin-4-yl]-amine and
(1-phenyl-1H-pyrazolo[3,4-d]pyrimidin-4-yl)-o-tolyl-amine,
or a pharmaceutically acceptable salt thereof, in the treatment, or for the manufacture of a pharmaceutical composition for the treatment, of a proliferative disease that depends on inadequate activity of a protein kinase, preferably a protein tyrosine kinase, especially one or more of c-Abl, c-Src and/or especially Ephrin receptor kinase, more especially EphB4 kinase; and/or one or more altered or mutated forms of any one or more of these, e.g. those forms that result in conversion of the respective proto-oncogene into an oncogene, such as constitutively activated Bcr-Abl or v-Src.

10. A compound of the formula I as shown in claim 1, wherein
R₁ is 5-fluoro-2-methylphenyl and 2-methylphenyl
R₂ is 4-lower alkoxyphenyl,
R₃ is hydrogen,
R₄ is hydrogen;
or a pharmaceutically acceptable salt thereof, for use in the diagnostic or therapeutic treatment of a warm-blooded animal, especially for use in the diagnostic and therapeutic treatment of a disease that depends on inadequate activity of a protein tyrosine kinase.

11. A compound of the formula I, wherein
R₁ is unsubstituted or substituted 3-nitrophenyl;
R₂ is substituted aryl;
R₃ is hydrogen or unsubstituted or substituted alkyl; and
R₄ is hydrogen or unsubstituted or substituted alkyl,
or a pharmaceutically acceptable salt thereof;
for use in the diagnostic or preferably therapeutic treatment of a warm-blooded animal, especially for use in the diagnostic and therapeutic treatment of a disease that depends on inadequate activity of a protein tyrosine kinase.

12. A process for the manufacture of a compound of the formula I, or a salt thereof, according to claim 4, comprising
reacting a pyrazolopyrimidine compound of the formula II, 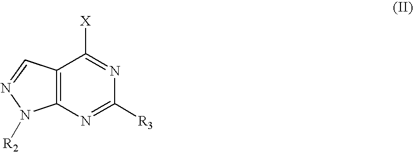
wherein R₂ and R₃ are as defined for a compound of the formula I and X is hydroxy or a leaving group, with an amino compound of the formula III,
H—NR₄—R₁ (III)
wherein R₁ and R₄ are as defined for a compound of the formula I;
and, if desired, transforming a compound of formula I into a different compound of formula I, transforming a salt of an obtainable compound of formula I into the free compound or a different salt, transforming an obtainable free compound of formula I into a salt thereof, and/or separating an obtainable mixture of isomers of a compound of formula I into individual isomers.

13. A pharmaceutical composition comprising a compound of the formula I, or a pharmaceutically acceptable salt thereof, according to claim 4 and a pharmaceutically acceptable carrier.

14. A compound of the formula I, or a pharmaceutically acceptable salt thereof, according to claim 4 for use in the diagnostic and/or therapeutic treatment of the animal, especially mammalian, or human body.

15. The use of a compound of the formula I, or a pharmaceutically acceptable salt thereof, according to any one of claims 4 to 7 in the treatment, or for the preparation of a pharmaceutical preparation for the treatment, of a disease or disorder that depends on inadequate activity of a protein kinase, preferably a protein tyrosine kinase, especially one or more of c-Abl, c-Src and/or especially Ephrin receptor kinase, more especially EphB4 kinase; and/or one or more altered or mutated forms of any one or more of these, e.g. those forms that result in conversion of the respective proto-oncogene into an oncogene, such as constitutively activated Bcr-Abl or v-Src.

16. The use of a compound of the formula I, or a pharmaceutically acceptable salt thereof, according to any one of claims 4 to 7 in the treatment, or for the manufacture of a pharmaceutical composition for the treatment, of a proliferative disease that depends on inadequate activity of a protein kinase, preferably a protein tyrosine kinase, especially one or more of c-Abl, c-Src and/or especially Ephrin receptor kinase, more especially EphB4 kinase; and/or one or more altered or mutated forms of any one or more of these, e.g. those forms that result in conversion of the respective proto-oncogene into an oncogene, such as constitutively activated Bcr-Abl or v-Src.

17. A method of treatment for a disease that responds to inhibition of a disease that depends on inadequate activity of a protein kinase, especially a protein tyrosine kinase; which comprises administering a prophylactically or especially therapeutically effective amount of a compound of formula I, or a pharmaceutically acceptable salt thereof, according to claim 1, especially to a warm-blooded animal, for example a human, that, on account of one of the mentioned diseases, requires such treatment.
